(19) United States
(12) Patent Application Publication
(10) Pub. No.: US 2007/0116698 A1
(43) Pub. Date: May 24, 2007

(54) Novel methods and medicament for treating infectious diseases involving microbial biofilms

(76) Inventor: Jean-Paul Perraudin, Brussel (BE)

Correspondence Address: AMRMSTRONG, KRATZ, QUINTOS,;HANSON & BROOKS, LLP, Suite 220, 502 Washington Avenue, Towson, MD, 21204, US

(21) Appl. No.: 11/480,676

(22) Filed: Jul. 3, 2006

Related U.S. Application Data
(63) Continuation-in-part of application No. 10/048,708, filed on Apr. 5, 2002, now abandoned.

(30) Foreign Application Priority Data
Jul. 7, 1999 (EP) 99870145.2

(51) Int. Cl. A61K 39/40; A61K 38/47; A61K 38/40; A61K 38/18; A61K 35/20; A61K 31/44; A61K 33/00

(52) U.S. Cl. 424/94.61; 424/164.1; 424/535; 424/661; 424/607; 514/12; 514/6; 514/358

(57) ABSTRACT

The present invention provides a method for prophylaxis and therapy of infectious diseases caused by microorganisms present in biofilms adherent to cell surfaces comprising the step of administering to an individual in need thereof a composition comprising a combination of at least one compound chosen from the group of peroxidase, lactoferrin, lactoferrin peptides, lysozyme and immunoglobulins and at least one growth factor.

### FIELD OF THE INVENTION

The present invention relates to the improvement of prophylactic and therapeutic applications of innate and non-innate defense mechanisms (peroxidases, lactoferrin, lactoferrin peptides, lysozyme, immunoglobulins, alone or combined) in the presence of growth factor proteins (platelet derived growth factor, fibroblast growth factor, transforming growth factor, epidermal growth factor, angiogenin, alone or combined) for the control or treatment of microorganisms, organized in biofilms, adherent to the cell surfaces.

### BACKGROUND OF THE INVENTION

The development of effective prophylactic and therapeutic agents for controlling microorganism biofilms adherent to cell surfaces has proven problematic.

Prophylactic and therapeutic formulations and methods developed for the prevention of infections by control of the ecological microbial balance, in general, have only been partially successful.

It is well known that natural antimicrobial agents are contained in most natural external mammalian secretions. In particular, the naturally occurring antimicrobial thiocyanate/peroxidase/H₂O₂ systems, lactoferrin, lactoferrin peptides, lysozyme and immunoglobulins present in secretion liquids have been extensively studied.

Antimicrobial thiocyanate/peroxidase/H₂O₂ systems produce hypothiocyanite (OSCN). These systems imitate the effect of the peroxidases (sialoperoxidase and myeloperoxidase) which catalyze the transformation of halide or pseudo-halide (as thiocyanate) into hypohalide or hypothiocyanite in the presence of the hydrogen peroxide produced by some bacterial strains.

Peroxidases and lactoperoxidase are known to stick to any support. There exists a paradox conflict between the in vitro cell toxicity and the in vivo clinical inefficiency data to decrease the bacterial count. Nevertheless, bacterial ATP content was shown in different species to decrease in vitro after halide and/or thiocyanate/peroxidase/H₂O₂ systems. This fact was confirmed in the human oral cavity after placement of a stick pill containing glucose/glucose-oxidase/thiocyanate/lactoperoxidase.

The resistance of oral mucosa to hypothiocyanite could be due to the protective role of biofilms on their surfaces. Some bacterial colonizers of these biofilms possess an NADH-hypothiocyanite-oxidoreductase (NHOR) activity, which can reduce hypothiocyanite. These bacteria with other strains produce hydrogen peroxide.

So biofilms containing H₂O₂ producers, can stick peroxidase and NHOR activity acts as a protective screen avoiding colonization by pathogenic microorganisms and preserving tissue integrity. In many other cases, in the presence of several layers of biofilms, the upper layer protects the lower layer against the action of the antibacterial agents. This lower layer containing H₂O₂ producers will thus be responsible for the damage of the tissue composed of epithelial and fibroblast cells.

In nutrient-limited ecosystems, such as the aquatic environment, bacteria have a marked tendency to attach to surfaces and initiate the formation of a biofilm. These biofilms are also a severe problem in medical science, such as in oral health where they can cause dental plaque and periodontitis.

The biofilm is a collection of microcolonies with water channels in between and an assortment of cells and extra cellular polymers (glycoproteins, polysaccharides and proteins). The ability of the antimicrobial agents contained in saliva to react significantly on the bacteria organized in biofilms depends largely on the thickness of the biofilm.

In fact some of these agents are able to bind the bacteria so avoiding the adhesion process on the mucosal cells. However, these antimicrobial agents are not able to remove the biofilm.

Biofilms predominate in the oral environment and it is troubling that the biofilm phenotype of some species has been shown to differ radically from the planktonic phenotype of the same organism. One of the facets in which biofilm bacteria differ the most profoundly from their planktonic counterparts, is in the critical matter of resistance to antibacterial agents.

Results of in vitro studies showed Staphylococcus epidermis and Staphylococcus aureus were significantly more sensitive to the Lactoperoxidase system where the microorganisms are under planktonic cells than the biofilm cells, since the number of viable planktonic cells decrease by approximately 6 log units compared to a reduction of 1 log units or less in the number of biofilm cells.

The test results on the total bacteria count confirm that biofilm cells are more resistant than planktonic cells. This is believed to be due to a physical protection by the biofilm matrix or by an altered physiology of bacterial mode of growth.

In some experiments, bacterial activity either by glucose oxidase or lactoperoxidase alone was observed. However, the daily variations in susceptibility of the biofilm cells may be explained by differences in the catalase activity and oxygen concentration.

In many cases, the bottom layer of biofilm will consist of anaerobic bacteria. As a result these biofilm cells may escape the inhibitory effect of the lactoperoxidase system, even though, under aerobic conditions, these cells have limited resistance to the lactoperoxidase system.

Moreover, the diffusion of thiocyanate and hydrogen peroxide into the biofilm will decrease the susceptibility of biofilm cells compared to planktonic cells. This suggests that the underlying cells of the biofilm will escape the antibacterial activity of the lactoperoxidase system unless the biofilms are released from the mucosal surface.

Such evidence may explain the difference in susceptibility between the bacteria in biofilm and the planktonic cells.

Lactoferrin is bacteriostatic by fixing ferric iron and making it unavailable for bacteria metabolism. Moreover lactoferrin presents a direct bactericidal effect on some microorganisms but as long as that the microorganisms are organized in biofilm and that the biofilm can be protected by other biofilm layers, the lactoferrin has no or not sufficient antibacterial effect against the lower layer.

It is the main object of the present invention to provide an effective composition against biofilms, i.e. which compositions are able to disinfect surfaces.

Lysozyme hydrolyses proteoglycans in the bacterial cell walls causing cell lysis. Lysozyme has a synergistic effect in combination with lactoferrin aggregating cell suspensions of some bacterial strains. As long as the microorganisms are organized in biofilms and are hidden by several layers of biofilms, lysozyme alone or in combination with lactoferrin has no effect against these microorganisms.

Immunoglobulins are able to react specifically against the microorganisms individually. The presence of several layers of biofilms and the characteristics of the biofilms avoid the action of the immunoglobulins alone or in combination with other antimicrobial agents such as lactoferrin to react against the individual microorganisms. These different innate and non-innate antimicrobial molecules present a synergetic effect in vitro on bacteria suspension.

These different innate and non-innate antimicrobial molecules present a synergetic effect in vitro on bacteria suspension but not on bacteria organized in biofilm.

Secretion liquids have long been known to be active against a number of bacteria, viruses, yeast and protozoa. But, saliva supplementation with thiocyanate/peroxidase/H₂O₂ systems has been shown to be ineffective in vivo on the salivary bacterial count. It could be stressed that methodology errors are at the basis of these contradictory data; the biological effects of antimicrobial agents were tested on planktonic bacteria suspended in saliva but not on bacteria organized in biofilms.

No formulation has been successful for the control of biofilms, limiting so the occurrence and progression of infectious diseases. Only antibiotics and other drugs inhibiting bioadhesion have been investigated.

Thus, it can be seen that there remains a need for prophylactic and therapeutic agents to be associated in synergy with molecules to improve the activity of the epithelial and fibroblast cells able to remove bacterial biofilms, thus yielding a better accessibility to the antimicrobial molecule for control of the growth and pathogenic potential of the microorganisms.

Growth factors are well known in the art. Among these growth factors, the platelet derived growth factor (PDGF), a dimeric protein, is able to stimulate the growth of the fibroblast cells. The fibroblast growth factor (FGF), monomeric protein is able also to stimulate the growth of the fibroblast cells. The transforming growth factor (TGF), presented by different polypeptides are able to stimulate the growth of the fibroblast cells and the epithelial cells. The epidermal growth factor (EGF) polypeptide is able to stimulate the growth of the epithelial cells. Angiogenic factors are able to stimulate the growth of endothelial cells.

### SUMMARY OF THE INVENTION

It was surprisingly shown by the present inventors that in association with antimicrobials, different growth factors are able to stimulate the growth of the fibroblast and/or epithelial cells and so to reactivate the activity of these cells destroyed by the binding of the pathogenic microorganisms organized in biofilms. This reactivation of the activity of fibroblast cells allows also the activation the epithelial cells which can be able by this action, to remove the microorganism biofilms from their cell surfaces. This removing of the microorganism biofilms yields them more accessible to the antibacterial, antiviral and candidacidal actions of the peroxidase/H₂O₂/Halide or pseudohalide, lactoferrin, lactoferrin peptides, lysozyme and immunoglobulins alone or combined.

In a first aspect, the present invention provides a composition comprising:

(b) at least one compound selected from lactoferrin, lactoferrin peptide, lysozyme, peroxidase system, immunoglobulins or a combination thereof, and

(c) at least one growth factor.

In second aspect, the present invention provides a composition comprising:

(a) at least one ion selected from hypohalite, hypothiocyanite or a combination thereof,

(b) at least one compound selected from lactoferrin, lactoferrin peptide, lysozyme, immunoglobulins or a combination thereof, and

(c) at least one growth factor.

The present invention also provides a pharmaceutical composition comprising a composition according to the invention, and a pharmaceutically acceptable carrier.

The present invention further provides a method for the prophylaxis and/or therapy of diseases caused by microorganisms comprising administering to an individual in need thereof a composition according to the invention. In particular, the method of the invention are for preventing or treating diseases selected from gastrointestinal infections, calculus and occurrence of caries, gingivitis, mucositis, periodontal diseases, vaginal diseases, herpes, acne, ulcers (E. pilori), tourista, cystitis, molluscum contagiosum or chlamydia infections, comprising the step of administering to an individual in need thereof a composition according to the invention.

The present invention further provides a method for the prophylaxis and therapy of infectious diseases caused by microorganisms present in biofilms adherent to cell surfaces comprising the step of administering to an individual in need thereof a composition according to the present invention.

It is a primary object of the present invention to provide uses (applications) for peroxidase system, lactoferrin, lactoferrin peptides, lysozymes and immunoglobulins (used alone and/or combined) for the control of microbial biofilms after the action of growth factors (used alone and/or combined) on the epithelial and fibroblast cells to remove microorganism biofilms adherent on their cell surfaces.

It is another primary object of the present invention to provide uses for peroxidase system, lactoferrin, lactoferrin peptides, lysozymes and immunoglobulins (used alone and/or combined) combined with growth factors (used alone and/or combined) in the preparation (or manufacture) of medicaments for the prophylaxis or therapy of disease caused by microorganism present in biofilms adherent on cell surfaces.

Lactoferrin peptides are peptides produced by the action of a protease or a combination of proteases on lactoferrin. For example said lactoferrin peptide is lactoferricin. Lactoferricin is a lactoferrin-derived peptide generated by acid-pepsin hydrolysis. Suitable lactoferrin peptides are described in Lactoferrin: Structure, Function and Applications. 2000. Elsevier, K. Simazaki et al. Editors. Citations can be found in particular on pages 164-171, on pages 239243, and on pages 273-277, incorporated herein by reference.

It is another object of the present invention to provide peroxidases, lactoferrin, lactoferrin peptides, lysozymes and immunoglobulins (used alone and/or combined) combined with growth factors preferably chosen from platelet derived growth factor, fibroblast growth factor, transforming growth factor, angiogenin and epidermal growth factor (used alone and/or combined) in the preparation (or manufacture) of medicaments for the prophylaxis or therapy of disease caused by microorganisms present in biofilms adherent on cell surfaces.

It is another object of the present invention to provide a peroxidase system (peroxidase) combined with a growth factor preferably chosen from platelet derived growth factor, fibroblast growth factor, transforming growth factor, angiogenin and epidermal growth factor (used alone and/or combined) in the preparation (or manufacture) of medicaments for the prophylaxis or therapy of disease caused by microorganisms present in biofilms adherent on cell surfaces.

It is another object of the present invention to provide lactoferrin or lactoferrin peptides combined with a growth factor preferably chosen from platelet derived growth factor, fibroblast growth factor, transforming growth factor, angiogenin and epidermal growth factor (used alone and/or combined) in the preparation (or manufacture) of medicaments for the prophylaxis or therapy of disease caused by microorganisms present in biofilms adherent on cell surfaces.

It is another object of the present invention to provide lysozyme combined with a growth factor preferably chosen from platelet derived growth factor, fibroblast growth factor, transforming growth factor, angiogenin and epidermal growth factor (used alone and/or combined) in the preparation (or manufacture) of medicaments for the prophylaxis or therapy of disease caused by microorganisms present in biofilms adherent on cell surfaces.

It is another object of the present invention to provide an immunoglobulin combined with a growth factor preferably chosen from platelet derived growth factor, fibroblast growth factor, transforming growth factor, angiogenin and epidermal growth factor (used alone and/or combined) in the preparation (or manufacture) of medicaments for the prophylaxis or therapy of disease caused by microorganisms present in biofilms adherent on cell surfaces.

It is another object of the present invention to provide peroxidase system, lactoferrin, lactoferrin peptides, lysozymes and immunoglobulins (used alone and/or combined) combined with platelet derived growth factor in the preparation (or manufacture) of medicaments for the prophylaxis or therapy of disease caused by microorganisms present in biofilms adherent on cell surfaces.

It is another object of the present invention to provide peroxidase system, lactoferrin, lactoferrin peptides, lysozymes and immunoglobulins (used alone and/or combined) combined with fibroblast growth factor in the preparation (or manufacture) of medicaments for the prophylaxis or therapy of disease caused by microorganisms present in biofilms adherent on cell surfaces.

It is another object of the present invention to provide peroxidase system, lactoferrin, lactoferrin peptides, lysozymes and immunoglobulins (used alone and/or combined) combined with transforming growth factor in the preparation (or manufacture) of medicaments for the prophylaxis or therapy of diseases caused by microorganisms present in biofilms adherent on cell surfaces.

It is another object of the present invention to provide peroxidase system, lactoferrin, lactoferrin peptides, lysozymes and immunoglobulins (used alone and/or combined) combined with epidermal growth factor in the preparation (or manufacture) of medicaments for the prophylaxis or therapy of disease caused by microorganisms present in biofilms adherent on cell surfaces.

It is another object of the present invention to provide peroxidase system, lactoferrin, lactoferrin peptides, lysozymes and immunoglobulins (used alone and/or combined) combined with angiogenin in the preparation (or manufacture) of medicaments for the prophylaxis or therapy of disease caused by microorganisms present in biofilms adherent on cell surfaces.

A further object of the present invention is to provide a composition comprising colostrum and/or whey colostrum for providing a source of said at least one growth factors.

It is another primary object of the present invention to provide prophylactic and therapeutic methods for controlling, preventing or treating infections in caused by microorganisms present in biofilms adherent on cell surfaces, by the administration of prophylactic and therapeutic effective amounts of peroxidases, lactoferrin, lactoferrin peptides, lysozymes and immunoglobulins (used alone and/or combined) and of prophylactic and therapeutic effective amounts of growth factors (used alone and/or combined) as defined above to individuals in need thereof.

As used herein, the term “prophylactic” refers variously to medicaments, amounts or quantities, methods, uses and effects, etc., that prevent and/or aid in preventing infections caused by the presence of microorganisms organized in biofilms adhered to cell surfaces. As used herein, the term “therapeutic” refers variously to medicaments, amounts or quantities, methods, uses and effects, etc., that ameliorate infections caused by the presence of microorganisms organized in biofilms adhered to cell surfaces.

These and other objects of the invention will become apparent from the following specification.

### DETAILED DESCRIPTION OF THE INVENTION

In a first embodiment, the formulations (or medicaments) of the present inventions include peroxidase system, lactoferrin, lactoferrin peptides, lysozyme and immunoglobulins on the one side and include proteins binding, by a means of a specific receptor, the epithelial and/or fibroblast cells and promoting growth of said cells such as for instance platelet derived growth factor (PDGF), fibroblast growth factor (FGF), transforming growth factor (TGF), angiogenin and epidermal growth factor (EGF) on the other side.

In a second embodiment, compositions of the present invention include hypohalite and/or hypothiocyanite, together with at least one compound selected from lactoferrin, lactoferrin peptides, lysozyme and immunoglobulins on the one side and at least a protein binding, by a means of a specific receptor, the epithelial and/or fibroblast cells and promoting growth of said cells such as for instance platelet derived growth factor (PDGF), fibroblast growth factor (FGF), transforming growth factor (TGF), angiogenin and epidermal growth factor (EGF) on the other side.

Preferably said hypohalite is selected from hypochlorite, hypoiodite, hypobromite ions or a combination thereof. In an embodiment, said composition is in a fluid state, such as liquid and comprises (a) at least one ion selected from hypochlorite, hypoiodite, hypobromite or hypothiocyanite or a combination thereof together with (b) at least one compound selected from lactoferrin, lactoferrin peptides, lysozyme, immunoglobulins or a combination thereof and (c) at least one growth factor selected from platelet derived growth factor, fibroblast growth factor, transforming growth factor, angiogenin, epidermal growth factor or a combination thereof. Preferably said hypohalite and/or hypothiocyanite is provided in a concentration ranging from 0.1 μM to 5000 μM, for example from 1 μM to 1000 μM, from 5 μM to 900 μM, from 5 μM to 800 μM from 5 μM to 700 μM, from 5 μM to 600 μM, from 5 μM to 550 μM, from 10 μM to 550 μM, from 20 μM to 550 μM, from 30 μM to 550 μM, from 40 μM to 550 μM.

The hypohalite and/or hypothiocyanite are used isolated and can be chemically produced. They can also be isolated from their producing enzymatic system using different process such as removing the enzyme from the producing solution, using an immobilization system, precipitating the enzymatic system or by using ultrafiltration membranes. The hypohalite and/or hypothiocyanite are preferably produced using an enzymatic process by reacting at least an enzyme of the peroxidase type with an oxidizable substrate and an oxygen donor, according to which i) an aqueous reaction mix is prepared comprising in addition to the oxidizable substrate and the oxygen donor, the enzyme in solid and divided phase, ii) aqueous reaction mix is fractioned in a fraction enriched with the enzyme in solid and divided phase, and a fraction free of said enzyme containing said hypohalite and/or hypothiocyanite ions.

A suitable production process is described in WO 02/097076, which recitations relating to the production process are hereby incorporated herein as part hereof. Said process comprises the steps of bringing into contact at least: one enzyme of the peroxidase type, and one oxidizable substrate, an oxygen donor, according to which: a) an aqueous reaction bath is formed, which bath comprises, in addition to the oxidizable substrate and the oxygen donor, said peroxidase in the solid and divided phase, but in the free state, b) the aqueous reaction bath is separated into a fraction which is enriched in enzyme in solid and divided phase and into a fraction which lacks said enzyme and from which the hypohalite and/or hypothiocyanite is obtained. The process can have the following variants: The oxidizable substrate, possibly in aqueous phase, is introduced into the aqueous reaction bath. The enzyme, in the state of a solid and divided phase or in liquid phase, is introduced into the reaction bath. The enzyme is discharged from the reaction bath. The process can be performed continuously or discontinuously. Aggregates may be used which are formed of solid particles inert with respect to the enzyme, said aggregates comprising or incorporating, in a free state, said enzyme, said aggregates being distributed in the aqueous reaction mix. Said aggregates may be flakes, obtained by introducing into the reaction mix, a flocculating agent. The flocculating agents which can be used are selected from the group comprising anion or cation polymeric flocculating agents, such as polysaccharides, heteropolysaccharides or polyacrylamines. Flocculating agents can be added in the reaction mix, in proportions varying from 0.1 10⁻⁶ to 10 g/l of reaction mix. Said aggregates may be coagulation products obtained by introducing into the reaction mix a coagulating agent. In an embodiment, a thickening agent may be introduced into the reaction mix. Coagulating agents can be chosen for example among iron or aluminum salts, such as for example: sulphate aluminum, chloride aluminum, sodium aluminate, aluminum polyhydroxychlorure, aluminum polyhydroxysultfate, polyhydroxychlorosultfate of aluminum, aluminum polychlorosulfate basic, aluminum polyhydroxychlorosilicate, aluminum fluorosulfate, sulphates ferrous, ferric sulphate, ferric chloride, chlorosulfate ferric, soda, or of the chloride homopolymers dimethyl diallyl ammonium. Coagulating agents can be added in the reaction mix, in proportions varying from 0.1 10⁻⁶ to 10 g/l of reaction medium.

The enzyme in solid phase can also in the form of an emulsion in the reaction mix, into which an emulsifying agent can be added. As used herein “emulsion” refers to any suspension containing an non-immobilized enzyme, likely to be obtained by addition of emulsifiers such as for example fatty substances or soya lecithin or hydrocarbons. The enzyme can also be comprised in a microorganism expressing said enzyme.

As used herein the term “aggregate” refers to any formulation allowing to maintain in a solid and divided phase but in a free state in the reaction mix the enzyme by addition of coagulating, flocculating, emulsifying agent in presence or not of a thickening agent, which allowing the isolation and the recycling of said enzyme from the reaction mix at the end of the reaction. The formation of the aggregates can be carried out by successive addition of a coagulating agent and of a flocculating agent.

As used herein “free state” refers to a state wherein said enzyme is comprised in said aggregate without formation of ionic or covalent bond between said enzyme and the aggregating agent. Optionally organics or mineral thickening agent, chemically compatible with the aggregates can be added with the mixture. This thickening agent can be introduced into the mix simultaneously with the introduction of the enzyme or after formation of the aqueous reaction mix. This thickening agent can be selected among clays, kaolin, silica, silicates, or any other compatible mineral agent. The thickening agent can be added in the reaction medium, in proportions varying of 0.1 to 100 g/l of reaction medium.

The pH of the reaction mix containing the enzyme, can be stabilized or corrected by addition of a pH correcting agent, which will be selected among mineral or organic acids or bases.

The hypohalite and/or hypothiocyanite can be produced in a reactor comprising a compartmentalized tank which can be partly or totally closed, and which can be made of metal or of synthetic material, said tank comprising a loading orifice, overflows and/or siphoid partitions, allowing the passage from one compartment to another.

The reactor may comprise three or four compartments, two or three of which are subjected to permanent stirring: the first compartment is intended to receive the enzyme, optionally the coagulating and the thickening agent, it is subjected to an agitation at fast speed, the second optionally receives the flocculating agent and optionally a pH corrector, and is under gentle stirring, the oxidation reaction is performed in the third compartment, wherein the oxidizable substrate as well as oxygen donor are added under gentle stirring. In the fourth compartment, the separation of the hypohalite and/or hypothiocyanite ions from the reaction mix is performed using a lamellar clarifier comprising a low feed, an overflow connected to the exit orifice, a low point of dynamic extraction of the settled solid matters.

Alternatively, the enzyme and the optional emulsifying agent are introduced into the first compartment, and the oxidation reaction is performed in the second compartment into which the oxidizable substrate is added. The third compartment is a recovery unit, which allows the uninterrupted pumping of the solution comprising hypohalite and/or hypothiocyanite ions and other residual substrates towards a separation unit, which can be a coalescor, a flotator, a centrifugal term “pseudo-halogens” refers to monovalent negatively charged ions selected from the group comprising thiocyanate ions, bisulfite ions, hydrosulphite ions, metabisutfite ions, and/or nitrite ions and the like. The oxidizable substrates suitable herein will be preferably selected according to the peroxidase used. Examples of suitable substrates include but are not limited to thiocyanate salts, such as sodium thiocyanate, potassium thiocyanate, ammonium thiocyanate, ferric thiocyanate. sodium bisulfite (NaHSO₃), sodium hydrosulfite (Na₂S₂O₄), sodium metabisulfite (Na₂S₂O₅), sodium nitrite (NaNO₂) or potassium nitrite (KNO₂), sodium hypochlorite (NaOCl) and potassium iodide (KI) and mixtures thereof.

The immobilized peroxidases which can be used to produce the hypohalite and/or hypothiocyanite ions for use in the present invention, include plant (vegetable) peroxidases, such as horseradish peroxidase, and mammalian peroxidases, such as salivary peroxidases, lactoperoxidases, myeloperoxidases and eosinophil peroxidase. These peroxidases may be extracted (isolated) from natural material, for instance mammalian material (for example, saliva, human and bovine milk) or produced by natural of chemical methods, all of which are well known to those skilled in the art. These peroxidases also include those peroxidases that are produced by recombinant DNA techniques, also well known in the art. Human and bovine lactoperoxidase may for instance be produced by microorganisms (for example transformed Pichia or transgenic animals such as transgenic cows) carrying a cDNA expressing said protein.

Depending on the hypohalite and/or hypothiocyanite to be produced, different enzymatic systems can be used. For example, salivary peroxidase can be used to produce hypothiocyanite, hypoiodite ions and/or mixtures thereof by using as oxidizable substrate thiocyanate, iodide and/or mixtures thereof in the presence of hydrogen peroxide. Lactoperoxidase can be used to produce hypothiocyanite, hypoiodite ions and/or mixtures thereof by using as oxidizable substrate thiocyanate, iodide and/or mixtures thereof in the presence of hydrogen peroxide. Myeloperoxidase can be used to produce hypothiocyanite, hypoiodite, hypochlorite ions and/or mixture thereof by using as oxidizable substrate thiocyanate, chloride, iodide ions and/or mixtures thereof, in the presence of hydrogen peroxide. Horseradish peroxidase can be used to produce hypoiodite, hypochlorite ions an/or mixture thereof by using as oxidizable substrate chlorides ions, iodides ions and/or mixtures thereof in the presence of hydrogen peroxide. Plant peroxidase can be used to produce hypothiocyanite, hypochlorite, hypobromite, hypoiodite ions and/or a mixture thereof by using as oxidizable substrate thiocyanates, chloride bromides iodide and/or mixture thereof in the presence of hydrogen peroxide.

Non-limiting examples of suitable peroxidases and/or oxidoreductases include enzymes of vegetable origin such as for example horseradish peroxidase (E.C. n° 1.11.1.7) or soya peroxidase, nitrate oxidoreductase-NADPH (E.C. n° 1.6.6.1) of cereals, enzymes from fungi such as for example glucose oxidase (E.C. n° 1.1.3.4), catalase (E.C. n° 1.11.1.6), betagalactosidase (E.C. n° 3.2.1.23) and nitrate oxydo-reductase-NADPH (E.C. n° 1.6.6.2) of Aspergillus, enzymes of bacterial origin such as for example NADH-Peroxidase (E.C. n° 11.1.1) of Enterococcus, NADPH oxidoreductase (E.C. n° 1.6.99.3) of Vibrio, nitrate reductase (E.C. n° 1.9.6.1) of Colibacilli, oxidase lactique dismutase (E.C. n° 1.1.3.2) of Pediococcus, superoxide dismutase (E C n° 1.15.1.1) of Colibacilli, Arthromyces peroxidase (E C n° 1.11.1.1), the betagalactosidase (E C n° 3.2.1.23) of Colibacilli, enzymes of animal origin such as for example xanthineoxidase (E.C. n° 1.1.3.22) of milk, lactoperoxidase (E.C. n° 1.11.1.7) of milk, the leucocytic myeloperoxidase (E.C. n° 1.11.1.7), nitric oxide synthase (E.C. n° 1.14.13.39) of nervous tissues, the superoxyde dismutase (E.C. n° 1.15.1.1) of erythrocytes, sulphite oxidase (E.C. n° 1.8.3.1) of hepatocytes.

The enzymes can be added in the reaction medium, in proportions varying from 0.02 to 10 g/l of reaction medium.

The oxidizable substrates can be added in the reaction medium, in proportions varying from 0.05 mM to 15 mM per liter of reaction medium,

The oxygen donor according to the present invention can be hydrogen peroxide and any inorganic peroxide such as metal peroxides, for example the peroxide of magnesium or of sodium or organic peroxides such as benzyl peroxides or the urea peroxide, but also the peracetic acid, the potassium permanganate and percarbonates. Generally, any chemical compound likely to produce hydrogen peroxide can be used. Advantageously the oxygen donors are added in the reaction medium, in proportions varying from 0.05 mM to 15 mM per liter of reaction medium. Said oxygen donor can be in the form of a complementary enzymatic system which produces oxygen peroxide, which comprises an oxidizable substrate and an enzyme for example of the oxidoreductase type, specific of this substrate.

Non-limiting examples of enzymatic systems suitable for the production of the oxygen donor include glucose oxidase/glucose, galactose oxidase/galactose, urate oxidase/urate, choline oxidase/choline, glycine oxidase/glycine, glycollate oxidase/glycollate, L-sorbose oxidase/L-sorbose, amine oxidase/primary amine, glutamate oxidase/glutamate, alcohol oxidase/alcohol, NAD(P)H oxidase/NAD(P)H, superoxide dismutase/oxygen free radical.

The above-mentioned enzymatic systems are able in the presence of oxygen and water to produce hydrogen peroxide which will be used as oxygen donor in the enzymatic system in the process for producing hypohalite and/or hypothiocyanite ions. Alternatively, the oxygen donor can be selected among hydrogen peroxide-producing-microorganisms such as Streptococcus and/or Lactobacillus.

In an embodiment, the present invention provides a composition comprising

a peroxidase system and

at least one growth factor preferably selected from platelet derived growth factor, fibroblast growth factor, transforming growth factor, angiogenin, epidermal growth factor or a combination thereof.

As used herein the term “peroxidase” or “peroxidase system” are used interchangeably and refers to peroxidase enzyme together with its oxidizable substrate and optionally a hydrogen donor. Preferably, the peroxidase component of the present invention includes a peroxidase/oxidizable substrate/hydrogen peroxide donor system that exhibits antiviral, antibacterial and candidacidal properties. In these formulations, peroxidase catalyses oxidation of the substrates (a halogen or pseudo-halogen) by a peroxide to form negatively charged, monovalent oxidizing compounds.

The substrates of the formulations of the present invention are chosen from a group consisting of negatively charged halogens, and their derivatives, and negatively charged pseudo-halogens, and their derivatives. The term “halogens” refers to certain of those elements, in their negatively-charged monovalent states, that belong to Group VII of the Periodic Table of Elements and, as is well known to those skilled in the art, includes bromide, chloride and iodide. The term “pseudo-halogens” refers to certain negatively charged ions and ionic compounds that are monovalent.

The “pseudo-halogens” of the present invention include the thiocyanate salts, such as sodium thiocyanate, potassium thiocyanate, ammonium thiocyanate, ferric thiocyanate and mixtures thereof.

The peroxidases present in the medicaments of the present invention include plant (vegetable) peroxidases, such as horseradish peroxidase, and mammalian peroxidases, such as salivary peroxidases, lactoperoxidases, myeloperoxidases and eosinophil peroxidase. These peroxidases may be extracted (isolated) from natural material (for example, saliva, human and bovine_milk) or produced by natural of chemical methods, all of which are well known to those skilled in the art. These peroxidases also include those peroxidases that are produced by recombinant DNA techniques, also well known in the art. Human and bovine lactoperoxidase may for instance be produced by microorganisms (for example transformed Pichia or transgenic animals such as transgenic cows) carrying a cDNA expressing said protein.

Examples of the preferred peroxidase/substrate combinations to be used in medicaments according to the present invention are also well known in the art. Examples of combinations are for instance referred to in U.S. Pat. No. 4,564,519 and U.S. Pat. No. 4,576,817, the contents of which are hereby incorporated by reference.

As utilized herein, the term “International Unit(s)” identifies that amount of the enzyme that will effect catalysis of 1 micromole of substrate per minute at pH 7 and 25° C. Enzymes are supplied in dry or liquid form with the label specifying the concentration in IU's on a per gram or per milliliter basis, as appropriate.

The peroxidases present in formulations destined for administration to young animals may also comprise metallic peroxide donors, such as magnesium peroxide and sodium carbamide peroxide such as described in European patent application published under No. 0 290 410 in name of Ewos Aktiebolag.

The substrates of these peroxidases and their derivatives can be extracted (isolated) from natural material (for example, saliva and human milk and vegetables) or produced by natural of chemical methods, all of which are well known to those skilled in the art.

Examples of the preferred peroxidase/substrate combinations to utilize in the medicament of the present invention are set forth below in Table IA:

TABLE IA

| Peroxidase | Substrates |
|---|---|
| Salivary peroxidase | Thiocyanate, iodide |
| Lactoperoxidase | Thiocyanate, iodide |
| Myeloperoxidase | Chloride, iodide, thiocyanate |
| Horseradish peroxidase | Chloride, iodide |
| Plant peroxidase | Chloride, iodide, bromide |

The reactions of representative enzyme systems from Table IA (in the presence of a peroxide—which for purposes of illustration herein, will be hydrogen peroxide—from the oxygen donor) to produce either a hypohalite or hypothiocyanite compound, are set forth in Table IB, as follows:

TABLE IB
(1a) Salivary peroxidase catalyzes the interaction of thiocyanate and hydrogen peroxide to produce
hypothiocyanite and water;
(1b) Salivary peroxidase catalyzes the interaction of iodide and hydrogen peroxide to produce
hypothiocyanite and water;
(2a) Lactoperoxidase catalyzes the interaction of thiocyanate and hydrogen peroxide to produce
hypothiocyanite and water;
(2b) Lactoperoxidase catalyzes the interaction of iodide and hydrogen peroxide to produce
hypothiocyanite and water;
(3a) Myeloperoxidase catalyzes the interaction of chloride and hydrogen peroxide to produce
hypochlorite and water;
(3b) Myeloperoxidase catalyzes the interaction of iodide and hydrogen peroxide to produce
hypoiodite and water;
(3c) Myeloperoxidase catalyzes the interaction of thiocyanate and hydrogen peroxide to produce
hypothiocyanite and water;
(4a) Horseradish peroxidase catalyzes the interaction of chloride and hydrogen peroxide to
produce hypochlorite and water;
(4b) Horseradish peroxidase catalyzes the interaction of iodide and hydrogen peroxide to produce
hypochlorite and water;
(5a) Plant peroxidase catalyzes the interaction of chloride and hydrogen peroxide to produce
hypochlorite and water;
(5b) Plant peroxidase catalyzes the interaction of iodide and hydrogen peroxide to produce
hypoiodite and water; and
(5c) Plant peroxidase catalyzes the interaction of bromide and hydrogen peroxide to produce
hypobromite and water.

The oxygen donor of the present invention provides (supplies) the peroxide (for example, hydrogen peroxide) in the medicament necessary for oxidation of the substrate.

Preferably, the oxygen donor is an enzymatic system including a substrate, an enzyme specific to such substrate and other necessary reactants, such as water and/or oxygen and/or hydrogen.

Alternatively, microorganisms, such as the Streptococci and Lactobacilli that are commonly referred to as lactic acid bacteria may be utilized in the medicaments of the present invention to supply the peroxide (in the form of hydrogen peroxide). Specific examples of such lactic acid bacteria include Lactobaccillus casei and Streptococcus faecalis and Streptococcus mutans. Use of such microorganisms (microbes) is especially preferred in the medicaments formulated for use as a vaginal cream for topical application.

It is also contemplated herein that inorganic peroxides (such as sodium peroxide and magnesium peroxide) or organic peroxides (such as benzyl peroxide and urea peroxide) may be utilized. Also, chemicals that, upon reaction, produce hydrogen peroxide may be utilized. Indeed, even hydrogen peroxide itself may be utilized as the oxygen donor. The precise oxygen donor to be utilized will vary depending upon several factors, including the formulation into which the medicament is to be made for administration.

Most preferably, the oxygen donor is an enzymatic system including an oxidizable substrate, an oxidoreductase enzyme specific to such substrate and other necessary reactants, such as oxygen and/or water. Examples of such oxidizable substrates, and oxidoreductase enzymes specific therefor, include those enumerated in U.S. Pat. No. 4,564,519 issued to Pellico et all. Such examples are set forth below in Table IIA:

TABLE IIA

| Oxidizable substrate | Oxidoreductase enzyme | Other reactants |
|---|---|---|
| B-D-glucose | Glucose oxidase | Water, Oxygen |
| D-galactose | Galactose oxidase | Oxygen |
| Urate | Urate oxidase | Water, Oxygen |
| Choline | Choline oxidase | Oxygen |
| D-amino acids¹ | D-amino acid oxidase | Water, Oxygen |
| D-glutamate | D-glutamate oxidase | Water, Oxygen |
| Glycine | Glycine oxidase | Water, Oxygen |
| Glycollate | Glycollate oxidase | Water, Oxygen |
| L-sorbose | L-sorbose oxidase | |
| Primary alcohol | Alcohol oxidase | |
| Primary amine | Amine oxidase | |
| NAD(P)H | NAD(P)H oxidase | |
| (m) Oxygen free radical | Superoxide dismutase | |

¹D-amino acids includes D isomers of proline, methionine, isoleucine, alanine, valine and phenylalanine.

The reactions of representative enzyme systems from Table IIA to produce hydrogen peroxide are set forth in Table IIB.

TABLE IIB
Glucose oxidase catalyzes the interaction of Beta-D-glucose, water and oxygen to produce
hydrogen peroxide and gluconic acid;
Galactose oxidase catalyzes the interaction of D-galactose and oxygen to produce hydrogen
peroxide and D-galacto-hexo-dioldose;
Urate oxidase catalyzes the interaction of urate, water and oxygen to produce hydrogen
peroxide, allantoin and carbon dioxide;
Choline oxidase catalyzes the interaction of choline and oxygen to produce hydrogen peroxide
and betaine aldehyde;
D-amino acid oxidase catalyzes the interaction of D-amino acids, such as the D-isomers of
proline, methionine, isoleucine, alanine, valine and phenylalanine together with water and oxygen
to produce hydrogen peroxide, ammonia and the corresponding alpha-keto acids;
D-glutamate oxidase catalyzes the interaction of D-glutamate, water and oxygen to produce
hydrogen peroxide, ammonia and 2-oxoglutarate; and
Glycine oxidase catalyzes the interaction of glycine, water and oxygen to produce hydrogen
peroxide, ammonia and glyoxylic acid.

The characteristics of representative oxidoreductase enzymes identified in Table IIA, from specific sources, are recited in U.S. Pat. No. 4,564,519, which recitations relating to these characteristics are hereby incorporated herein as part hereof.

Most preferably, the peroxidase medicaments of the present invention include either lactoperoxidase or myeloperoxidase in combination with a thiocyanate (SCN—) substrate and of a glucose/glucose oxidase enzymatic system oxygen donor.

It is preferred that the above-mentioned peroxidase/substrate/peroxide systems be formulated into the prophylactic and therapeutic medicaments for “in vivo” use as a substantially self-contained system that may be applied or used substantially without depending upon the users naturally-occurring “in vivo” concentrations of substrate, oxygen donors, peroxidases or other ingredients.

It is noted that the effectiveness of the peroxidase medicaments of the present invention may be effected by the naturally-occurring environment in which the medicament is to be administered. For example, in the human mouth, the concentration of hydrogen peroxide varies as a direct function of biological production and salivary flow. When salivary flow is at a diminished level, either as a natural event or as an event arising out of certain types of medical treatment, the oral concentrations of various elements, such as potassium thiocyanate and peroxidase, will be correspondingly reduced. This, in turn, may be a limiting factor in the prophylactic or therapeutic effectiveness of the medicament when it is orally administered. Moreover, when the oral concentration of peroxidase is suppressed through diminished salivary flow, oral concentrations of hydrogen peroxide may increase to a threshold level, wherein the hydrogen peroxide can impede the effectiveness of peroxidase of the medicament.

Accordingly, it can be seen that the concentrations of the substrate, oxygen donor and peroxidase (peroxidase system) in the medicaments described above should be adjusted and controlled to harmonize hydrogen peroxide and peroxidase concentration, so as to limit the hydrogen peroxide concentrations to levels which do not impede with the activity of the peroxidase.

As utilized herein, the term millimole identifies that quantity in grams corresponding to the molecular weight of the medicament divided by one thousand.

When the oxygen donor is hydrogen peroxide itself, it is generally present in the medicament of the present invention in an amount from about 2 to about 300 millimoles per gram of per milliliter of medicament, for example 3 to 200 millimoles per gram of per milliliter of medicament, 3 to 100 millimoles per gram of per milliliter of medicament, 3 to 70 millimoles per gram of per milliliter of medicament, 3 to 60 millimoles per gram of per milliliter of medicament, 3 to 50 millimoles per gram of per milliliter of medicament, and, preferably, from about 3 to about 30 millimoles per gram or per milliliter of medicament.

In the event the oxygen donor is an oxidizable substrate and an oxidoreductase enzyme specific to the substrate, then the oxidizable substrate is generally present in the peroxidase medicament in an amount from about 0.015 to about 0.6 millimole per gram or per milliliter of medicament, for example from 0.015 to 0.5 millimole per gram or per milliliter of medicament, from 0.015 to 0.4 millimole per gram or per milliliter of medicament, from 0.015 to 0.3 millimole per gram or per milliliter of medicament, from 0.015 to 0.2 millimole per gram or per milliliter of medicament, from 0.02 to 0.1 millimole per gram or per milliliter of medicament, and, preferably, from about 0.025 to about 0.1 millimole per gram or per milliliter of medicament while the oxidoreductase is generally present in the medicament in an amount from about 0.5 to about 500 IU's per gram or per milliliter of the medicament and, preferably, from about 1.0 to about 40 IU per gram or per milliliter of the medicament.

In the event the oxygen donor is an organic or inorganic peroxide, then such peroxide is generally present in the medicament in an amount from about 0.000006 to about 0.6 millimole per gram-or per milliliter of medicament and, preferably, from about 0.00006 to about 0.6 millimole per gram or per milliliter of medicament.

The substrate is generally present in the medicament in an amount ranging from about 0.0000008 to about 0.01 millimole per gram of per milliliter of medicament and, preferably, from about 0.000008 to about 0.006 millimole per gram of per milliliter of medicament.

In the event that the substrate is a thiocyanate salt (a pseudo-halogen), then it is generally present in the medicament in an amount from about 0.0001 to about 0.01 millimole per gram or per milliliter of medicament and, preferably, from about 0.001 to about 0.006 millimole per gram or per milliliter of medicament. Care should be taken in formulating the medicament, so as to avoid the use of metal compounds which inhibit or impair the effectiveness of the enzymes.

In the event that the substrate is a halogen, then it is generally present in the medicament in an amount from about 0.0000008 to about 0.008 millimole per gram or milliliter per medicament and, preferably, from about 0.000008 to about 0.004 millimole per gram of per milliliter of medicament.

The peroxidase is generally present in the medicaments in an amount from about 0.01 to about 50 IU per gram of per milliliter of medicament and, preferably, in an amount from about 0.2 to about 4.0 IU per gram or per milliliter of medicament.

It is noted that, if desired, the peroxidase medicament may be formulated for “in vivo” use as a system that relies upon certain naturally-occurring “in vivo” concentrations of any one or combination of compounds of the system for obtaining the peroxidase-generated compound.

The antiviral prophylactic and therapeutic qualities of the peroxidase medicaments of the present invention may be dependent on the concentration of compounds that are produced by the formulation of the medicament of the present invention. The produced concentrations of these compounds may vary between 1 micro molar and 100 millimolar, with concentrations of between 5 micro molar and 1 millimolar being preferred. For achieving this, the concentrations of the oxygen donor and/or of the substrate is able to be varied over a large range.

The presence of water promotes the oxidation/reduction reactions of the peroxidase medicaments of this invention. It also is a reactant in certain reactions. Thus, preferably, the use of water in formulating the said medicaments should be at a relatively low concentration levels in order to impart maximum stability and shelf life thereto.

Where the products of the activated enzyme systems in the medicaments include a weak organic acid, it is advantageous to formulate the medicament with a buffering agent to neutralize the organic acid. A suitable buffering agent is sodium bicarbonate.

In this regard, it is preferred that the peroxidase medicaments of the present invention should be formulated, so as to have a pH that substantially approximates physiological pH. In particular, it is preferred that the medicaments of the present invention have a pH ranging from 4.5 tot 6.5, with a pH of from 6 to 6.5 being especially preferred.

In an embodiment, the present invention provides a composition comprising:

a lactoferrin and

at least one growth factor preferably selected from platelet derived growth factor, fibroblast growth factor, transforming growth factor, angiogenin, epidermal growth factor or a combination thereof.

In another embodiment, the present invention provides a composition comprising:

a lactoferrin,

at least one ion selected from hypochlorite, hypobromide, hypoiodite, hypothiocyanite or a combination thereof, and

at least one growth factor preferably selected from platelet derived growth factor, fibroblast growth factor, transforming growth factor, angiogenin, epidermal growth factor or a combination thereof.

In the formulations according to the present invention, lactoferrin, presented under different iron saturated forms from 0% of iron (apo-lactoferrin) to 100% of iron saturation (Iron saturated lactoferrin) can be provided from different sources induding mammalian sources for instance bovine lactoferrin from bovine secretions liquids for example bovine milk, human lactoferrin from human secretion liquids for example human milk, cDNA human like-lactoferrin or bovine like-lactoferrin produced by microorganisms for example Pichia or from transgenic animals for example transgenic cows, all known in the art. In an embodiment, lactoferrin is provided in a concentration ranging from 0.1 μg to 5000 mg/l of composition or from 0.1 μg to 5000 mg/kg of composition. Preferred dosages ranges of lactoferrin are from 0.001 g to 10 g, preferably from 0.01 g to 0.1 g per kg bodyweight per day or per 100 ml of liquid, gel, paste or other formulation.

In an embodiment, the present invention provides a composition comprising:

lactoferrin peptides, and

at least one growth factor preferably selected from platelet derived growth factor, fibroblast growth factor, transforming growth factor, angiogenin, epidermal growth factor or a combination thereof.

In another embodiment, the present invention provides a composition comprising:

lactoferrin peptides,

at least one ion selected from hypochlorite, hypobromide, hypoiodite, hypothiocyanite or a combination thereof, and

at least one growth factor preferably selected from platelet derived growth factor, fibroblast growth factor, transforming growth factor, angiogenin, epidermal growth factor or a combination thereof.

Lactoferrin peptides are peptides produced by the action of a protease or a combination of proteases on lactoferrin. The proteases can be pepsin, chymotrypsin or any of all other proteases (proteolytic enzymes) used alone or in combined to proteolyse lactoferrin from any source such as described hereabove. For example said lactoferrin peptide is lactoferricin. Lactoferricin is a lactoferrin-derived peptide generated by acid-pepsin hydrolysis. Suitable lactoferrin peptides are described in Lactoferrin: Structure, Function and Applications. 2000. Elsevier, K. Simazaki et al. Editors, pages 164-171, on pages 239-243, and on pages 273-277, and in Advances in Lactoferrin Research, Edited by Spik et al. Plenum Press, 1998, on page 285-291, incorporated herein by reference.

In an embodiment, lactoferrin peptide is provided in a concentration ranging from 0.1 μg to 5000 mg/l of composition or from 0.1 μg to 5000 mg/kg of composition. Useful concentration can be from 10 μg to 10 g of lactoferrin peptides per liter but preferably 100 μg to 100 mg of lactoferrin peptides per liter per kg bodyweight per day or per 100 ml of liquid, gel, paste or other formulation.

In an embodiment, the present invention provides a composition comprising:

lysozyme, and

at least one growth factor preferably selected from platelet derived growth factor, fibroblast growth factor, transforming growth factor, angiogenin, epidermal growth factor or a combination thereof.

In another embodiment, the present invention provides a composition comprising:

lysozyme,

at least one ion selected from hypochlorite, hypobromide, hypoiodite, hypothiocyanite or a combination thereof, and

at least one growth factor preferably selected from platelet derived growth factor, fibroblast growth factor, transforming growth factor, angiogenin, epidermal growth factor or a combination thereof.

In these formulations, lysozyme can be provided from different sources including mammalian sources for instance bovine lysozyme from bovine secretion liquids for example bovine milk, human lysozyme from human secretion liquids for example human milk, egg white lysozyme from egg white for example hen egg white, cDNA human like-lysozyme or bovine like-lysozyme produced by microorganisms for example Pichia or from transgenic animals for example transgenic cows. In an embodiment, lysozyme is provided in a concentration ranging from 0.1 μg to 5000 mg/l of composition or from 0.1 μg to 5000 mg/kg of composition. Preferred dosages ranges of lysozyme are from 0.001 g to 50 g, preferably from 0.01 g to 10 g, more preferably from 0.01 g to 0.1 g per kg bodyweight per day or per 100 ml of liquid, gel, paste or other formulation.

In an embodiment, the present invention provides a composition comprising:

immunoglobulins, and

at least one growth factor preferably selected from platelet derived growth factor, fibroblast growth factor, transforming growth factor, angiogenin, epidermal growth factor or a combination thereof.

In another embodiment, the present invention provides a composition comprising:

immunoglobulins,

at least one ion selected from hypochlorite, hypobromide, hypoiodite, hypothiocyanite or a combination thereof, and

at least one growth factor preferably selected from platelet derived growth factor, fibroblast growth factor, transforming growth factor, angiogenin, epidermal growth factor or a combination thereof.

In these formulations, immunoglobulins can be provided from different sources including mammalian sources for instance bovine immunoglobulins from bovine secretions liquids for example blood, colostrum, milk and other derivates, human immunoglobulins from human secretion liquids for example blood, milk and other derivates, egg immunoglobulins from egg yolk. That undertakes also immunoglobulins produced from secretion liquids of immunized animals. Purified immunoglobulin preparations known in the art and commercially available may also be used. In an embodiment, immunoglobulin is provided in a concentration ranging from 0.1 μg to 100 g/l of composition or from 0.1 μg to 100 g/kg of composition. Preferred dosages ranges of immunoglobulins are from 0.001 g to 1000 g, preferably from 0.001 g to 100 g, more preferably from 0.01 g to 10 g, most preferably from 0.05 g to 1 g per kg bodyweight per day or per 100 ml of liquid, gel, paste or other formulations.

The use of different of these above-mentioned compounds together has a synergetic effect.

In these formulations, the growth factor component can be provided from any source known in the art. Preferred dosages ranges of growth factors are from 1 ppb to 100 mg, preferably from 0.001 mg to 100 mg, more preferably from 0.01 mg to 10 mg, most preferably from 0.1 mg to 1 g per kg bodyweight per day or per 100 ml of liquid, gel, paste or other formulations.

In an embodiment, the present invention provides a composition comprising:

at least one compound selected from peroxidases, lactoferrin, lactoferrin peptides, lysozymes, immunoglobulins or a combination thereof, and

platelet derived growth factor.

In another embodiment, the present invention provides a composition comprising:

at least one ion selected from hypochlorite, hypobromide, hypoiodite, hypothiocyanite or a combination thereof,

at least one compound selected from lactoferrin, lactoferrin peptides, lysozymes, immunoglobulins or a combination thereof, and

platelet derived growth factor.

In these formulations, the platelet derived growth factor can be provided from mammalian platelets for instance from human or pig platelets or from bovine and human secretions liquids for example colostrum, milk and other derivates, manufactured by recombinant DNA technique, chemically synthesized, or a mixture thereof, all known in the art.

In an embodiment, the present invention provides a composition comprising:

at least one compound selected from peroxidases, lactoferrin, lactoferrin peptides, lysozymes, immunoglobulins or a combination thereof, and

fibroblast growth factor.

In another embodiment, the present invention provides a composition comprising:

at least one ion selected from hypochlorite, hypobromide, hypoiodite, hypothiocyanite or a combination thereof,

at least one compound selected from lactoferrin, lactoferrin peptides, lysozymes, immunoglobulins or a combination thereof, and

fibroblast growth factor.

In these formulations, the fibroblast growth factor can be provided from hypophyse, brain, hypothalamus, retin surrenal gland, and kidney. He can also provide from mammalian secretion for instance from bovine and human secretions liquids for example colostrum, milk and other derivates, manufactured by recombinant DNA techniques, chemically synthesized, or a mixture thereof, all known in the art.

In an embodiment, the present invention provides a composition comprising:

at least one compound selected from peroxidase system, lactoferrin, lactoferrin peptides, lysozymes, immunoglobulins or a combination thereof, and

transforming growth factor.

In another embodiment, the present invention provides a composition comprising:

at least one ion selected from hypochlorite, hypobromide, hypoiodite, hypothiocyanite or a combination thereof,

at least one compound selected from lactoferrin, lactoferrin peptides, lysozymes, immunoglobulins or a combination thereof, and

transforming growth factor.

In these formulations, the transforming growth factor can be provided from pig blood platelet, from mammalian blood platelet, preferably, bovine and human secretions liquids for example colostrum, milk and other derivates, manufactured by recombinant DNA techniques, chemically synthesized, or a mixture thereof, all known in the art.

In an embodiment, the present invention provides a composition comprising:

at least one compound selected from peroxidases, lactoferrin, lactoferrin peptides, lysozymes, immunoglobulins or a combination thereof, and

epidermal growth factor.

In another embodiment, the present invention provides a composition comprising:

at least one ion selected from hypochlorite, hypobromide, hypoiodite, hypothiocyanite or a combination thereof,

at least one compound selected from lactoferrin, lactoferrin peptides, lysozymes, immunoglobulins or a combination thereof, and

epidermal growth factor.

In these formulations, the epidermal growth factor can be provided from different tissues and biological liquids of mammalian species, from bovine and human secretions liquids for example colostrum, milk and other derivates, manufactured by recombinant DNA techniques, chemically synthesized, or a mixture thereof, all known in the art.

In an embodiment, the present invention provides a composition comprising:

at least one compound selected from peroxidases, lactoferrin, lactoferrin peptides, lysozymes, immunoglobulins or a combination thereof, and

angiogenin.

In another embodiment, the present invention provides a composition comprising:

at least one ion selected from hypochlorite, hypobromide, hypoiodite, hypothiocyanite or a combination thereof,

at least one compound selected from lactotenin, lactoferrin peptides, lysozymes, immunoglobulins or a combination thereof, and

angiogenin.

In these formulations, the angiogenin growth factor can be provided from different tissues and biological liquids of mammalian species, from bovine and human secretions liquids for example colostrum, milk and other derivates, manufactured by recombinant DNA techniques, chemically synthesized, or a mixture thereof, all known in the art.

The use of different of these above-mentioned growth factors together has a synergetic effect.

In an embodiment, the present invention provides a composition comprising colostrum and/or whey colostrum for providing a source of said at least one growth factors. In an embodiment, the present invention provides a composition comprising: (a) -at least one ion selected from hypochlorite, hypobromide, hypoiodite, hypothiocyanite or a combination thereof, and (b) at least one first compound selected from lactoferrin, lactoferrin peptides, lysozymes, immunoglobulins or a combination thereof, and (c) at least one second compound selected from colostrum, whey colostrum, platelet derived growth factor, fibroblast growth factor, transforming growth factor, angiogenin, epidermal growth factor or a combination thereof.

In an embodiment, the present invention provides a hypohalite and/or hypothiocyanite and a lactoferrin or lactoferrin peptide combined with colostrum and/or whey colostrum.

In an embodiment, the present invention provides a hypohalite and/or hypothiocyanite and a lysozyme combined with colostrum and/or whey colostrum.

In an embodiment, the present invention provides a hypohalite and/or hypothiocyanite and an immunoglobulin combined with colostrum and/or whey colostrum.

The above mentioned compositions may further comprise a compound selected from triclosan, chlorhexidine, cetylpyridinium, or a combination thereof. Chlorhexidine can be chemically or naturally produced chlorhexidine. Preferred concentrations of chlorhexidine for use herein are ranging from 0.1 μg to 100 g/l. Triclosan can be chemically or naturally produced. Preferred concentrations of triclosan for use herein are ranging from 0.1 μg to 100 g/l. Cetylpyridinium can be chemically or naturally produced. Preferred concentrations of Cetylpyridinium for use herein are ranging from 0.1 μg to 100 g/l.

In a preferred embodiment, the composition according to the invention comprises a combination of at least three compounds: a first compound selected from the group comprising lactoferrin peptides, lactoferrin, lysozyme, immunoglobulins or a combination thereof, a second compound selected from the growth factors and a third compound selected from the group of ions comprising OSCN-(hypothiocyanite), Ol-(hypoidoite) or OCl (hypochlorite) or a combination thereof.

In the present invention, the hypohalite and/or hypothiocyanite ions used in the composition are isolated and not in combination with the peroxidase system which usually comprises the enzyme, the substrate and the oxygen donor.

The hypohalite and/or hypothiocyanite ions when isolated have an activity which is different from the ion when in presence of the peroxidase system.

The different possible formulations according to the present invention may be prepared for prophylactic and/or therapeutic purposes, as desired and needed, for permitting the administration of prophylactic and/or therapeutic effective amounts of the individual components thereof to an individual in need thereof for preventing and/or treating infections.

The present invention provides methods for the prophylaxis and/or therapy of diseases caused by microorganisms comprising administering to an individual in need thereof a composition according to the invention. In an embodiment, said method comprises administering to an individual in need thereof a composition comprising (a) at least one compound selected from lactoferrin, lactoferrin peptide, lysozyme, peroxidase system, immunoglobulins or a combination thereof, and (b) at least one growth factor.

In an alternative embodiment, the present invention also provides a method for the prophylaxis and/or therapy of diseases caused by microorganisms comprising administering to an individual in need thereof a composition comprising (a) at least one ion selected from hypohalite or hypothiocyanite or a combination thereof, (b) at least one compound selected from lactoferrin, lactoferrin peptide, lysozyme, immunoglobulins or a combination thereof, and (c) at least one growth factor.

Said microorganisms can be bacteria, viruses, protozoas, yeast, fungi, parasites and the like.

In an embodiment, said microorganism is responsible of gastrointestinal infections. In an embodiment said gastro intestinal infection is causing diarrhea or intestinal ulcers. Preferably said microorganism responsible of gastrointestinal infection is selected from the group comprising Shigella, Salmonella, E. coli, Vibreo colera, Pseudomonas (Ps. pyocyanea), Staphylococcus (Staph. albus, aureus), Streptococcus (Strep. vindans, Strep. faecalis, B-Streptococcus), Proteus, Helicobacter pilori and the like, preferably H. pilori.

In another embodiment, said microorganism is involved in the formation of calculus and occurrence of caries, gingivitis, mucositis and/or other periodontal diseases. In another embodiment, said microorganism is causing acne. In another embodiment, said microorganism is responsible of vaginal diseases. In another embodiment, said microorganism is responsible of molluscum contagiosum.

In a further embodiment, said microorganism is an enveloped virus. For example, said virus is causing herpes. Preferably said herpes is herpes simplex paramyxoviruses (such as human parainfluenza viruses), the family of orthomyxoviruses (such as the influenza type viruses A and B), rotaviruses, coronaviruses, herpes viruses (such as Varicella-zoster virus, cytomegalovirus, Epstein-Barr virus and HHV6) and retroviruses (such as Human T-cell leukemia virus-1, bovine leukemia virus and simian immunodeficiency virus SIV))).

The formulation according to the present invention may be used to prevent and/or treat infections in humans or animals.

The formulations according to the present invention can be used for the prophylaxis or therapy of infectious diseases caused by microorganisms forming biofilms on various types of human cell surfaces, such as for instance skin, ocular mucosa, orthorhino-laryngic spheres, gastro-enterologic cells and cell surfaces of the urogenital system.

The formulations according to the present invention are for instance useful for treating dental plaque, periodontal diseases, ulcers, tourista, bacterial vaginitis, vaginosis, cystitis, molluscum contagiosum, chlamydia infections.

The medicaments according to the present invention may be administrated in any form known in the art, and are for instance in the form of a topical medicament, an oral dentifrice or an injectable composition, and preferentially in the form of a gel, a stick pill, a rinsing liquid or a toothpaste, tablets, soft gelatin capsules, lozenges, powder mixtures, etc.

The pharmaceutical compositions or medicaments according to the present invention, and for use in accordance to the present invention, may comprise, in addition to the aforementioned compounds, a pharmaceutically acceptable excipient, carrier, buffer, stabilizer or other materials well known to those skilled in the art. Such materials should be non-toxic and should not interfere with the efficacy of the active ingredients. The precise nature of the carrier or other material may depend on the route of administration. Those of relevant skill in the art are well able to prepare suitable solutions.

In a therapeutic context, i.e. where the biological effect of the formulations to an individual is beneficial, administration is preferably in a “therapeutically effective amount”, this being sufficient to show benefit to the patient. Such benefit may be at least amelioration of one symptom. The actual amount administered, and rate and time-course of the administration, will depend on the aim of the administration, e.g. the biological effect sought in view of the nature and severity of the challenge and is the subject of routine optimization. Prescription of treatment, for example decisions on dosage etc., is within the responsibility of general practitioners and other medical doctors.

The medicaments of the present invention will be better understood by reference to the following examples, which are illustrative only and are not meant to be limiting in any manner.

### BRIEF DESCRIPTION OF THE DRAWINGS

FIG. 1 represents the bacterial count expressed in percentage in function of the time of incubation upon using stick pill formulations 1, 2 and 3 as detailed in Example V.

FIG. 2 represents the ATP percentage in function of the time of incubation upon using stick pill formulations 1, 2 and 3 as detailed in Example V.

FIG. 3 represents the ratio of ATP/bacterial count in function of the time of incubation upon using stick pill formulations 1, 2 and 3 as detailed in Example V.

FIG. 4 represents the bacterial count (CFU) in function of the time after brushing with toothpaste formulations 1, 2 and 3 as detailed in Example VI.

FIG. 5 represents the ATP in function of the time after brushing with toothpaste formulations 1, 2 and 3 as detailed in Example VI.

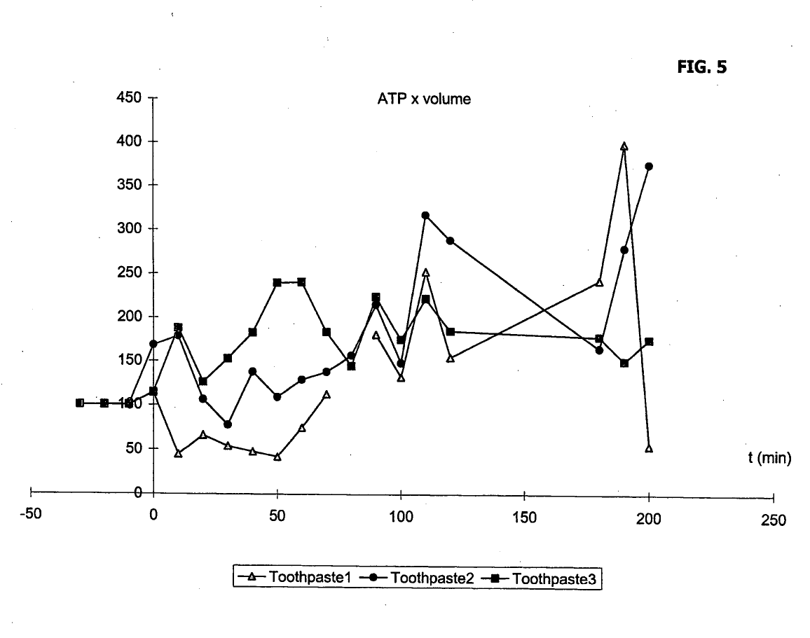

FIG. 6 represents the ATP/bacterial count (CFU) ratio in function of the time after brushing with the toothpaste formulations 1, 2 and 3 as detailed in Example VI.

### EXAMPLES

Production of OSCN⁻, or OI⁻ or OCI⁻ ion

The peroxidase immobilized on a solid support is added to a beaker. H₂O₂ and SCN⁻ or I⁻ or Cl⁻ or a mixture of two or three thereof is added.

The solid support can be sludge, glass beads or chromatographic cationic supports and the like. The enzyme can be covalently fixed. The enzyme can be tonically fixed or fixed by absorption.

Once the reaction over, the mixture is settled and filtrated to remove the immobilized enzyme.

The filtrated solution contains OSCN⁻ and/or or OI⁻ and/or ocr. As used herein these solutions will be referred as “OSCN⁻ solutions, “OI⁻ solution∞, “OCl⁻ solutions, OSCN⁻/Ol⁻/OCl⁻ solution, OSCN⁻/OI⁻ solution, OCl⁻/OI⁻ solution, OSCN⁻/OCl⁻ solution. The solution may also contain unreacted substrate.

The hypohalite and/or hypothiocyanite ions produced herein have an activity against microorganism having sulfhydryl groups on their membrane. However, in the presence of its producing enzymatic system (continuous production) the ion can also react with the tyrosine presents in the proteins, enzymes and peptides present in the medium. Iodination of protein (marker) for tumor detection is known. The iodination mechanism is only possible with a peroxidase system and not with the OI-alone.

The hypohalite and/or hypothiocyanite ions when together with their enzymatic production system tend to react not only with microorganisms but also with tyrosine present in molecules, which diminish the effective concentration of said ions.

When these ions are used without their enzymatic system, they only react on the microorganisms since the reaction with tyrosine is only catalyzed in the presence of the peroxidase.

The same phenomenon can be observed in the presence of unsaturated oils, which double bond can be saturated with the above mentioned ions only in the presence of their enzymatic production system. Such saturated oil presents a rancid taste. The saturation reaction does not happen when the ions are isolated and used without their enzymatic production system.

Another example of the difference of activity of the above mentioned ions when isolated can be shown with hemoglobin which can be oxidized only in the presence of peroxidase system. This phenomenon does not happen when the ions are used without their enzymatic producing system.

### Example I

Illustrative base formulations for pharmaceutically-acceptable carriers for the peroxidase medicaments to be formulated with as a dentifrice for oral administration, such as a chewing gum and chewable tablets and lozenges are set forth in Table III, as follows:

TABLE III

Weight, Percent

| Ingredients | (a) | (b) | (c) | (d) |
|---|---|---|---|---|
| Sorbitol, crystalline | 75 | — | 98 | 28 |
| Corn sugar | — | 75 | — | 70 |
| Gum base | 23 | 23 | — | — |
| Flavor | 1 | 1 | 1 | 1 |
| Color | 0.5 | 0.5 | 0.5 | 0.5 |
| Buffer | — | — | 0.5 | 0.5 |
| Saccharin, sodium | 0.005 | — | 0.005 | — |

In table III, formulations (a) and (b) illustrate pharmaceutically-acceptable carriers in the form of chewing gum compositions while formulations (c) and (d) illustrate pharmaceutical-acceptable carriers in the form of tablet and lozenge compositions. Aspartame can be substituted for sodium saccharin in these formulations.

The following examples show varying ingredients and concentration levels which can be used in the preparation of dentifrices for providing the prophylactic and therapeutic effective amounts for oral administration according to the present invention:

TABLE IV

| Chewing gum: | Weight, Grams | | | | | |
|---|---|---|---|---|---|---|
| Ingredients | 4A | | 4B | | 4C | |
| Sorbitol | 70 | | 70 | | 70 | |
| Gum base | 23 | | 23 | | 23 | |
| Glycerol | 5 | | 5 | | 5 | |
| Flavor | 1 | | 1 | | 1 | |
| Color | 0.5 | | 0.5 | | 0.5 | |
| Sodium Bicarbonate | 0.5 | | 0.5 | | 0.5 | |
| | 100 | | 100 | | 100 | |
| Enzymes: | | | | | | |
| Glucose Oxidase | 40,000 | IU | | | | |
| B-D Glucose | 1.0 | g | | | | |
| Choline Oxidase | | | 8000 | IU | | |
| Choline | | | 1.0 | g | | |
| D-glutamate Oxidase | | | | | 2,500 | IU |
| D-glutamate | | | | | 0.1 | g |
| Lactoperoxidase | 4,000 | IU | 1,500 | IU | 1,000 | IU |
| Potassium thiocyanate | 0.01 | g | 0.005 | g | | |
| Sodium thiocyanate | | | 0.01 | g | | |
| Lactoferrin | 0.1 | g | 0.1 | g | 0.1 | g |
| Lysozyme | 0.1 | g | 0.1 | g | 0.1 | g |
| Immunoglobulins | 1 | g | 1 | g | 1 | g |
| Growth factors | | | | | | |
| Derived Platelet growth factor | 0.01 | mg | 0.01 | mg | 0.01 | mg |
| Transforming growth factor | 0.005 | mg | 0.005 | mg | 0.005 | mg |
| Fibroblast growth factor | 0.01 | mg | 0.01 | mg | 0.01 | mg |
| Epidermal growth factor | 0.015 | mg | 0.015 | mg | 0.015 | mg |
| Angiogenin | 0.001 | mg | 0.001 | mg | 0.001 | mg |

TABLE V

| Chewing gum: | Weight, Grams | | | | | |
|---|---|---|---|---|---|---|
| Ingredients | 5A | | 5B | | 5C | |
| Sorbitol, Cryst. | 43 | | 43 | | 43 | |
| Gum base | 20 | | 20 | | 20 | |
| Glycerol | 25 | | 25 | | 25 | |
| Flavor | 1 | | 1 | | 1 | |
| Color | 0.5 | | 0.5 | | 0.5 | |
| Sodium Bicarbonate | 0.5 | | 0.5 | | 0.5 | |
| | 100 | | 100 | | 100 | |
| Enzymes: | | | | | | |
| D-amino acid oxidase | 5,000 | IU | | | | |
| D-alanine | 0.1 | g | | | | |
| Glucose oxidase | | | 20,000 | IU | 2,000 | IU |
| B-D-Glucose | | | 0.5 | g | 0.5 | g |
| Lactoperoxidase | 4,000 | IU | 1,500 | IU | 1,000 | IU |
| Potassium thiocyanate | 0.01 | g | 0.005 | g | | |
| Sodium thiocyanate | | | | | 0.01 | g |
| Lactoferrin | 0.1 | g | 0.1 | g | 0.1 | g |
| Lysozyme | 0.1 | g | 0.1 | g | 0.1 | g |
| Immunoglobulins | 1 | g | 1 | g | 1 | g |
| Growth factors | | | | | | |
| Derived Platelet growth factor | 0.01 | mg | 0.015 | mg | 0.005 | mg |
| Transforming growth factor | 0.005 | mg | 0.0025 | mg | 0.01 | mg |
| Fibroblast growth factor | 0.015 | mg | 0.01 | mg | 0.005 | mg |
| Epidermal growth factor | 0.015 | mg | 0.01 | mg | 0.005 | mg |
| Angiogenin | 0.001 | mg | 0.001 | mg | 0.001 | mg |

TABLE VI

| Lozenges | Weight, Grams | | | | | |
|---|---|---|---|---|---|---|
| Ingredients | 6A | | 6B | | 6C | |
| Sorbitol, Crystalline | 97 | | 97 | | 97 | |
| Glycerol | 1 | | 1 | | 1 | |
| Flavor | 1 | | 1 | | 1 | |
| Color | 0.5 | | 0.5 | | 0.5 | |
| Sodium Bicarbonate | 0.5 | | 0.5 | | 0.5 | |
| | 100 | | 100 | | 100 | |
| Enzymes | | | | | | |
| Glucose Oxidase | 10,000 | IU | | | | |
| B-D Glucose | 1.0 | g | | | | |
| Choline Oxidase | | | | | 2,000 | IU |
| Choline | | | | | 0.5 | g |
| Uranate oxidase | | | 10,000 | IU | | |
| Urate | | | 0.75 | g | | |
| Lactoperoxidase | 2,000 | IU | 2,000 | IU | 1,000 | IU |
| Potassium thiocyanate | | | | | 0.01 | g |
| Sodium thiocyanate | 0.01 | g | 0.01 | g | | |
| Lactoferrin | 0.1 | g | 0.2 | g | 0.05 | g |
| Lysozyme | 0.1 | g | 0.2 | g | 0.05 | g |
| Immunoglobulins | 1 | g | 1 | g | 1 | g |
| Growth factors | | | | | | |
| Derived Platelet growth factor | 0.01 | mg | 0.01 | mg | 0.01 | mg |
| Transforming growth factor | 0.005 | mg | 0.005 | mg | 0.005 | mg |
| Fibroblast growth factor | 0.01 | mg | 0.01 | mg | 0.01 | mg |
| Epidermal growth factor | 0.015 | mg | 0.015 | mg | 0.015 | mg |
| Angiogenin | 0.001 | mg | 0.001 | mg | 0.001 | mg |

TABLE VII

| Lozenges: | Weight, Grams | | | | | |
|---|---|---|---|---|---|---|
| Ingredients | 7A | | 7B | | 7C | |
| Sorbitol, Crystalline | 80 | | 80 | | 80 | |
| Corn Sugar | 17 | | 17 | | 17 | |
| Flavor | 1 | | 1 | | 1 | |
| Color | 0.5 | | 0.5 | | 0.5 | |
| Sodium Bicarbonate | 0.5 | | 0.5 | | 0.5 | |
| | 100 | | 100 | | 100 | |
| Enzymes: | | | | | | |
| D-glutamate oxidase | 10,000 | IU | — | | — | |
| D-glutamate | 0.05 | g | — | | — | |
| Glucose Oxidase | — | | 5,000 | IU | 1,000 | IU |
| B-D Glucose | — | | 0.5 | g | 1 | g |
| Lactoperoxidase | 1,500 | IU | 2,000 | IU | 1,000 | IU |
| Potassium thiocyanate | 0.001 | g | 0.005 | g | — | |
| Sodium thiocyanate | — | | — | | 0.005 | g |
| Lactoferrin | 0.1 | g | 0.1 | g | 0.1 | g |
| Lysozyme | 0.1 | g | 0.1 | g | 0.1 | g |
| Immunoglobulins | 1 | g | 1 | g | 1 | g |
| Growth factors | | | | | | |
| Derived Platelet growth factor | 0.01 | mg | 0.015 | mg | 0.005 | mg |
| Transforming growth factor | 0.005 | mg | 0.0025 | mg | 0.01 | mg |
| Fibroblast growth factor | 0.015 | mg | 0.01 | mg | 0.005 | mg |
| Epidermal growth factor | 0.015 | mg | 0.01 | mg | 0.005 | mg |
| Angiogenin | 0.001 | mg | 0.001 | mg | 0.001 | mg |

TABLE VIII

| Soft gelatin capsules: | Weight, Grams | | | | | |
|---|---|---|---|---|---|---|
| Ingredients | 8A | | 8B | | 8C | |
| Onager oil | 0.275 | g | — | | — | |
| Vit. C | 0.06 | g | 0.06 | g | 0.06 | g |
| Vit. E | 0.01 | g | 0.01 | g | 0.01 | g |
| Beta carotene | 0.0001 | g | 0.0001 | g | 0.0001 | g |
| Selenium | 0.0001 | g | 0.0001 | g | 0.0001 | g |

| | | | | |
|---|---|---|---|---|
| Soya oil | — | 0.275 | g | — |

| | | | | |
|---|---|---|---|---|
| Fish oil (23% DHA) | — | — | 0.275 | g |

Enzymes: per 800 mg soft gelatin capsules

| Superoxide dismutase | 100 | IU | 500 | IU | 1,000 | IU |
|---|---|---|---|---|---|---|
| Lactoperoxidase | 1,500 | IU | 2,000 | IU | 1,000 | IU |
| Potassium thiocyanate | 0.01 | g | 0.05 | g | — | |
| Sodium thiocyanate | — | | — | | 0.05 | g |
| Lactoferrin | 0.05 | g | 0.1 | g | 0.1 | g |
| Lysozyme | 0.05 | g | 0.1 | g | 0.1 | g |
| Immunoglobulins | 0.1 | g | 0.05 | g | 0.1 | g |

Growth factors

| | | | | | | |
|---|---|---|---|---|---|---|
| Derived Platelet growth factor | 0.01 | mg | 0.015 | mg | 0.005 | mg |
| Transforming growth factor | 0.005 | mg | 0.0025 | mg | 0.01 | mg |
| Fibroblast growth factor | 0.015 | mg | 0.01 | mg | 0.005 | mg |
| Epidermal growth factor | 0.015 | mg | 0.01 | mg | 0.005 | mg |
| Angiogenin | 0.001 | mg | 0.001 | mg | 0.001 | mg |

### Example II

Illustrative base formulations for pharmaceutically-acceptable carriers for the peroxidase medicaments of the present invention to be formulated as a topical medicament for topical administration, such as a cream, a gel or to be incorporated in a bandage or pad, are set forth in Table IX, as follows:

TABLE IX

| | |
|---|---|
| Gel | Weight, Percent |

| Ingredients | 9A | 9B |
|---|---|---|
| Deionized water | 19.02 | 20.0 |
| Corn Starch¹ | 38.04 | — |
| Lubrajel DV² | 38.04 | — |
| Aloe vera | 0.000021 | — |
| Natrosol 250 M³ | 0.1 | — |
| Xylitol | 4.76 | — |
| Cirami N.1⁴ | — | 20.0 |
| Sunflower Oil | — | 40.0 |
| Vitamin E | — | 0.05 |
| Tensami 4/07⁴ | — | 2.0 |
| Tensami 1/05⁴ | — | 3.0 |
| Bronopol⁴ | — | 2.0 |
| Myacide SP⁴ | — | 2.0 |
| Propylene Glycol | — | 10.0 |

¹An example of such a corn starch is the hydrogenated starch solution marketed under the name HYSTAR TPF by Alban Muller International, Montreuil, France.
²Lubragel DV is a Glycerin and acrylic solution marketed by Alban Muller International, Montreuil, France.
³Natrosol 250 M is a hydroxeyethycellulose marketed by Aqualon, Inc., of Hopewell, Virginia, U.S.A.
⁴Cirami N.1, Tensami 4/07, Tensami 1/05, Bronopol and Myacide SP are all marketed by Alban Muller International, Montreuil, France.

In table IX, formulation (a) illustrates pharmaceutically-acceptable carriers in the form of a gel, and (b) illustrates pharmaceutically-acceptable carriers in the form of a cream. The following tables show varying ingredients and prophylactic and therapeutic effective amounts (quantities) which can be used in the preparation of topical peroxidase medicaments, according to the present invention:

TABLE X

| | |
|---|---|
| Gel: | Weight, Grams |

| Ingredients | 10A | 10B |
|---|---|---|
| Deionized water | 19.03 | 19.03 |
| Corn Starch | 38.054 | 38.054 |
| Lubrajel DV | 38.054 | 38.054 |
| Aloe vera | 0.001 | 0.001 |
| Natrosol 250 M | 0.1 | 0.1 |
| Xylitol | 4.76 | 4.76 |
| | 100 | 100 |

Enzymes:

| Glucose Oxidase | 10,000 | IU | — | |
|---|---|---|---|---|
| Glucose | 1.0 | g | — | |
| Choline Oxidase | — | | 8,000 | IU |
| Choline | — | | 1.0 | g |
| Lactoperoxidase | 2,000 | IU | 1,500 | IU |
| Potassium thiocyanate | — | | 0.005 | g |
| Sodium thiocyanate | 0.05 | g | — | |
| Lactoferrin | 0.05 | g | 0.1 | g |
| Lysozyme | 0.05 | g | 0.1 | g |
| Immunoglobulins | 0.1 | g | 0.1 | g |

Growth factors

| | | | | |
|---|---|---|---|---|
| Derived Platelet growth factor | 0.01 | mg | 0.005 | mg |
| Transforming growth factor | 0.005 | mg | 0.01 | mg |
| Fibroblast growth factor | 0.015 | mg | 0.005 | mg |
| Epidermal growth factor | 0.015 | mg | 0.005 | mg |
| Angiogenin | 0.001 | mg | 0.001 | mg |

TABLE XIa

| Cream: | Weight, Grams | | |
|---|---|---|---|
| Ingredients | 11A | 11B | 11C |
| Deionized Water | 21.51 | 21.51 | 21.51 |
| Cirami N. | 20.0 | 20.0 | 20.0 |
| Sunflower Oil | 40.0 | 40.0 | 40.0 |
| Vitamin E | 0.04 | 0.04 | 0.04 |
| Tensami 4/07 | 2.0 | 2.0 | 2.0 |
| Tensami 1/05 | 3.0 | 3.0 | 3.0 |
| Bronopol | 2.0 | 2.0 | 2.0 |
| Myacide SP | 2.0 | 2.0 | 2.0 |
| Propylene Glycol | 10.0 | 10.0 | 10.0 |
| | 100 | 100 | 100 |

Enzymes:

| Glucose Oxidase | 5,000 | IU | — | | — | |
|---|---|---|---|---|---|---|
| Glucose | 0.5 | g | — | | — | |
| D-amino acid oxidase | — | | 5,000 | IU | — | |
| D-alanine | — | | 0.1 | g | — | |
| Urate oxidase | — | | — | | 10,000 | IU |
| Urate | — | | — | | 0.75 | g |
| Lactoperoxidase | 2,000 | IU | 1,000 | IU | 1,500 | IU |
| Potassium thiocyanate | 0.005 | g | — | | — | |
| Sodium thiocyanate | — | | 0.01 | g | 0.08 | g |
| Lactoferrin | 0.05 | g | 0.1 | g | 0.1 | g |
| Lysozyme | 0.05 | g | 0.1 | g | 0.1 | g |
| Immunoglobulins | 0.1 | g | 0.05 | g | 0.1 | g |

Growth factors

| | | | | | | |
|---|---|---|---|---|---|---|
| Derived Platelet growth factor | 0.01 | mg | 0.015 | mg | 0.005 | mg |
| Transforming growth factor | 0.005 | mg | 0.0025 | mg | 0.01 | mg |
| Fibroblast growth factor | 0.015 | mg | 0.01 | mg | 0.005 | mg |
| Epidermal growth factor | 0.015 | mg | 0.01 | mg | 0.005 | mg |
| Angiogenin | 0.001 | mg | 0.001 | mg | 0.001 | mg |

TABLE XIb
Unless otherwise provided the weights are given in grams

| Ingredients | A | | B | | C | |
|---|---|---|---|---|---|---|
| OSCN - (21.51% deionized water) | 250 | μM | — | | — | |
| OI - (21.51% deionized water) | — | | 200 | μM | — | |
| OCI - (21.51% deionized water) | — | | — | | 200 | μM |
| Cirami N. | 20.0 | | 20.0 | | 20.0 | |
| Sunflower Oil | 40.0 | | 40.0 | | 40.0 | |
| Vitamin E | 0.04 | | 0.04 | | 0.04 | |
| Tensami 4/07 | 2.0 | | 2.0 | | 2.0 | |
| Tensami 1/05 | 3.0 | | 3.0 | | 3.0 | |
| Bronopol | 2.0 | | 2.0 | | 2.0 | |
| Myacide SP | 2.0 | | 2.0 | | 2.0 | |
| Propylene Glycol | 10.0 | | 10.0 | | 10.0 | |
| | 100 | | 100 | | 100 | |
| Lactoferrin | 0.05 | g | 0.05 | g | — | |
| Lysozyme | 0.05 | g | — | | 0.05 | g |
| Immunoglobulins | 0.1 | g | 0.05 | g | 0.1 | g |

Growth factors

| | | | | | | |
|---|---|---|---|---|---|---|
| Derived Platelet growth factor | 0.01 | mg | 0.015 | mg | 0.005 | mg |
| Transforming growth factor | 0.005 | mg | 0.0025 | mg | 0.01 | mg |
| Fibroblast growth factor | 0.015 | mg | 0.01 | mg | 0.005 | mg |
| Epidermal growth factor | 0.015 | mg | 0.01 | mg | 0.005 | mg |
| Angiogenin | 0.001 | mg | 0.001 | mg | 0.001 | mg |

TABLE XII

| Cream: | Weight, Grams | | |
|---|---|---|---|
| Ingredients | 12A | 12B | 12C |
| Deionized Water | 21.51 | 21.51 | 21.51 |
| Cirami N. | 20.0 | 20.0 | 20.0 |
| Sunflower Oil | 40.0 | 40.0 | 40.0 |
| Vitamin E | 0.04 | 0.04 | 0.04 |
| Tensami 4/07 | 2.0 | 2.0 | 2.0 |
| Tensami 1/05 | 3.0 | 3.0 | 3.0 |
| Bronopol | 2.0 | 2.0 | 2.0 |
| Myacide SP | 2.0 | 2.0 | 2.0 |
| Propylene Glycol | 10.0 | 10.0 | 10.0 |
| | 100 | 100 | 100 |

Enzymes:

| | | | |
|---|---|---|---|
| Superoxide dismutase | 500 IU | 1,000 IU | 2,000 IU |
| Lactoperoxidase | 2,000 IU | 1,000 IU | 1,500 IU |
| Potassium thiocyanate | 0.005 g | — | — |
| Sodium thiocyanate | — | 0.01 g | 0.08 g |
| Lactoferrin | 0.05 g | 0.1 g | 0.1 g |
| Lysozyme | 0.05 g | 0.1 g | 0.1 g |
| Immunoglobulins | 0.1 g | 0.05 g | 0.1 g |

### Example III

Illustrative formulations for pharmaceutically-acceptable carriers for the peroxidase medicaments of the present invention to be formulated as an eye wash solution for topical administration, as an eye drop or as an eye wash, are set forth in Table XIII, as follows:

The following Table shows the varying ingredients and the prophylactic and therapeutic effective amounts (quantities) which can be used in the preparation of eye wash medicaments, according to the present invention:

TABLE XIII (a)

| | Weight, Percent | |
|---|---|---|
| Ingredients | 13A | 13B |

Eye drop or eye wash (per 5 ml solution)

| | | |
|---|---|---|
| Sorbic Acid 0.0025% | — | 0.0002 |
| Purified Water | 99.4 | 98.1 |
| Boric Acid | 0.018 | 0.0176 |
| Sodium Borate | 0.0015 | 0.0013 |
| (Hydrated 10 H₂O) | | |
| Sodium Chloride | 0.0025 | — |
| Benzalkonium chloride¹ | 0.0001 | — |
| Edetate disodium¹ | 0.001 | — |

Enzymes: (amounts per 5 ml Eye Wash¹)

| | | | | |
|---|---|---|---|---|
| Glucose oxidase | 2,500 | IU² | | |
| Glucose | | | 0.02 | g |
| Superoxide dismutase | | | 100 | IU |
| Lactoperoxidase | 200,000 | ABTS units³ | 150,000 | ABTS units³ |
| Potassium thiocyanate | | | 0.0005 | g |
| Sodium thiocyanate | | | 0.0005 | g |
| Lactoferrin | 0.0001 | g | 0.00005 | g |
| Lysozyme | 0.0001 | g | 0.00005 | g |
| Immunoglobulins | 0.0001 | g | 0.00005 | g |

Growth factors

| | | | | |
|---|---|---|---|---|
| Derived Platelet | 0.001 | mg | 0.0005 | mg |
| growth factor | | | | |
| Transforming growth | 0.0005 | mg | 0.0001 | mg |
| factor | | | | |
| Fibroblast growth | 0.0015 | mg | 0.0005 | mg |
| factor | | | | |
| Epidermal growth factor | 0.0015 | mg | 0.0005 | mg |
| Angiogenin | 0.0001 | mg | 0.0001 | mg |

¹Benzalkonium chloride and Edetate disodium are added as preservatives.

TABLE XIII (b)
Eye drop or eye wash (per 5 ml solution)

| Ingredients | | | |
|---|---|---|---|
| OSCN - (99.4% | 250 μM | — | — |
| deionized water) | | | |
| OI - (99.2% deionized | 250 μM | 200 μM | — |
| water) | | | |
| OCI - (98.1% deionized | — | — | 200 μM |
| water) | | | |
| Sorbic acid. (0.0025%) | 0.0002 | — | — |
| Boric acid | 0.018 | 0.018 | 0.018 |
| Sodium borate | 0.0015 | 0.0015 | 0.0015 |
| (hydrated 10H20) | | | |
| Sodium chloride | 0.0025 | 0.0025 | — |
| Benzalkonium chloride | 0.0001 | — | — |
| Edetate disodium | 0.001 | 0.001 | 0.001 |
| | 100 | 100 | 100 |
| Lactoferrin | 0.0005 g | 0.0005 g | — |
| Lysozyme | 0.0005 g | — | 0.0005 g |
| Immunoglobulins | 0.0001 g | 0.0001 g | 0.0001 g |

Growth factors

| | | | |
|---|---|---|---|
| Derived Platelet growth factor | 0.001 mg | 0.0015 mg | 0.0005 mg |
| Transforming growth factor | 0.0005 mg | 0.00025 mg | 0.0001 mg |
| Fibroblast growth factor | 0.00015 mg | 0.0001 mg | 0.00005 mg |
| Epidermal growth factor | 0.00015 mg | 0.0001 mg | 0.00005 mg |
| Angiogenin | 0.00001 mg | 0.00001 mg | 0.00001 mg |

¹The eye wash solution is a 5 ml solution of: 90 milligrams of Boric acid; 6.6 milligrams of hydrated sodium borate (10 H₂O); 2500 units Vitamin A and 0.125 μg of sorbic acid 0.0025%
²As utilized in this example, the term “unit” of Glucose oxidase identifies that amount of Glucose oxidase that oxidizes 3.0 milligram glucose to gluconic acid in one minute at pH 5.10 and 37° C. The assay conditions are set forth in Assay method FS 250 of Finnish Sugar Co. Ltd., of Finland. In this Example, 1 milligram of glucose oxidase has an activity of 100-120 units at 37° C. at pH5.
³As utilized herein, the term “ABTS units” identifies that amount of lactoperoxidase that catalyzes the oxidation of 1 mM of the ABTS substrate (2,2′-Azino-bis(3-Ethylbenzthiazoline-6-Sulfonic Acid)) in one minute at pH 5 and 37° C. The assay conditions are set forth by Mansson-Rahemtulla, B., et al., Biochemistry, Vol. 27, at pages 233-239 (1988). In this Example, 1 milligram of lactoperoxidase has an activity of 600 ABTS units at 37° C. and 5 pH.

In case of the formulation 13A, the composition is formulated separately in two parts which, before application, are combined and shaken to dissolve and mix the two parts.

The first part is a mixture of the lactoperoxidase and the glucose oxidase. The second part is a 5 ml solution of the boric acid, hydrated sodium borate (10 H₂O), Vitamin A, 0.0025% sorbic acid, potassium thiocyanate, water and glucose. The 5 ml solution (the second part) is mixed with the first part and shaken to dissolve the powder. Administration may be made as normal eye drops.

### Example IV

Illustrative formulations for pharmaceutically-acceptable carriers for the peroxidase medicaments of the present invention to be formulated as a tooth paste for pets and a feed complement for calves, are set forth in Table XIV and XV, as follows:

TABLE XIV

| | |
|---|---|
| Toothpaste for pets | |
| (per 100 g of paste) | Weight, Grams |

| Ingredients: | 14A | | 14B | |
|---|---|---|---|---|
| Malt | 10 | g | 10 | g |
| Sorbitol | 40 | g | 40 | g |
| Glycerin | 10 | g | 10 | g |
| Water | 2 | g | 2 | g |
| Xanthan Gum | 6 | g | 6 | g |
| Calcium diphosphate | 10 | g | 10 | g |
| Silica | 10 | g | 10 | g |
| Emulsifier | 12 | g | 12 | g |

Enzymes:

| Glucose oxidase | 10,000 | IU | 2,000 | IU |
|---|---|---|---|---|
| Glucose | 0.2 | g | 0.25 | g |
| Lactoperoxidase | 10,000 | | 15,000 | |
| | ABTS units (3) | | ABTS units (3) | |
| Potassium thiocyanate | | | 0.0005 | g |
| Sodium thiocyanate | | | 0.0015 | g |
| Lactoferrin | 0.01 | g | 0.005 | g |
| Lysozyme | 0.01 | g | 0.005 | g |
| Immunoglobulins | 0.1 | g | 0.05 | g |

Growth factors

| | | | | |
|---|---|---|---|---|
| Derived Platelet growth factor | 0.001 | mg | 0.0005 | mg |
| Transforming growth factor | 0.0005 | mg | 0.0001 | mg |
| Fibroblast growth factor | 0.0015 | mg | 0.0005 | mg |
| Epidermal growth factor | 0.0015 | mg | 0.0005 | mg |
| Angiogenin | 0.0001 | mg | 0.0001 | mg |

TABLE XV
Feed complements for calves
(per 100 g of powder)

| | Weight, Grams | | | |
|---|---|---|---|---|
| Ingredients: | 15A | | 15B | |
| Milk powder | 42.5 | g | 42.5 | g |
| Bovine colostrum | 42.5 | g | 42.5 | g |

Enzymes:

| Glucose oxidase | 12,000 | IU (2) | 10,000 | IU (2) |
|---|---|---|---|---|
| Glucose | 0.2 | g | 0.25 | g |
| Lactoperoxidase | 10,000 | | 15,000 | |
| | ABTS units (3) | | ABTS units (3) | |
| Potassium thiocyanate | | | 0.0005 | g |
| Sodium thiocyanate | | | 0.0015 | g |
| Lactoferrin | 0.01 | g | 0.005 | g |
| Lysozyme | 0.01 | g | 0.005 | g |
| Immunoglobulins | 12 | g | 15 | g |

Growth factors

| | | | | |
|---|---|---|---|---|
| Derived Platelet growth factor | 0.001 | mg | 0.0005 | mg |
| Transforming growth factor | 0.0005 | mg | 0.0001 | mg |
| Fibroblast growth factor | 0.0015 | mg | 0.0005 | mg |
| Epidermal growth factor | 0.0015 | mg | 0.0005 | mg |
| Angiogenin | 0.0001 | mg | 0.0001 | mg |

### Example V

This example shows the effectiveness of the enzyme group (Peroxidase/substrate/peroxide system—Lactoferrin-Lysozyme-Immunoglobulins) in combination with the growth factors (Platelet Derived growth factor—Fibroblast growth factor—Transforming growth factor—Epidermal growth factor—Angiogenin) of the present invention against oral microorganisms.

Stick Pills with active ingredients Enzymes and Growth Factors: Stick 1

Amounts per stick pill

| | | |
|---|---|---|
| Active Ingredients | | |
| Lactoperoxidase | 100 | IU |
| Glucose Oxidase | 26 | IU |
| B-D-Glucose | 25 | mg |
| Substrate (KI) | 1.1 | mg |
| Lactoferrin | 10 | mg |
| Lysozyme | 10 | mg |
| Immunoglobulins | 30 | mg |
| Platelet Derived growth factor | 10 | ng |
| Transforming growth factor | 12 | ng |
| Fibroblast growth factor | 5 | ng |
| Epidermal growth factor | 100 | ng |
| Angiogenin | 0.1 | ng |
| Non active ingredients | | |
| Carbopol 974P, | | |
| Drum-Dried-Waxy-Maire Starch | | |

Stick Pills with only the Enzymes as active ingredients: Stick 2

| | Amounts per stick pill | |
|---|---|---|
| Active Ingredients | | |
| Lactoperoxidase | 100 | IU |
| Glucose Oxidase | 26 | IU |
| Glucose | 25 | mg |
| Substrate (KI) | 1.1 | mg |
| Lactoferrin | 10 | mg |
| Lysozyme | 10 | mg |
| Immunoglobulins | 30 | mg |
| Non active ingredients | | |
| Carbopol 974P, Drum-Dried-Waxy-Maire Starch | | |

Stick Pills where the active ingredients is replaced by maltodextrin: Stick 3 (control)

Active Ingredients:
maltodextrin
Non active ingredients:
Carbopol 974P, Drum-Dried-Waxy-Maire Starch

The Stick Pills 1 to 3 were applied to patients suffering from a dry mouth because they produced less than 0.2 ml per minute of saliva. The Stick Pill is sticked to the gum and allows a slow release of the active ingredients during several hours in the mouth of the patients. 24 patients were used in each group.

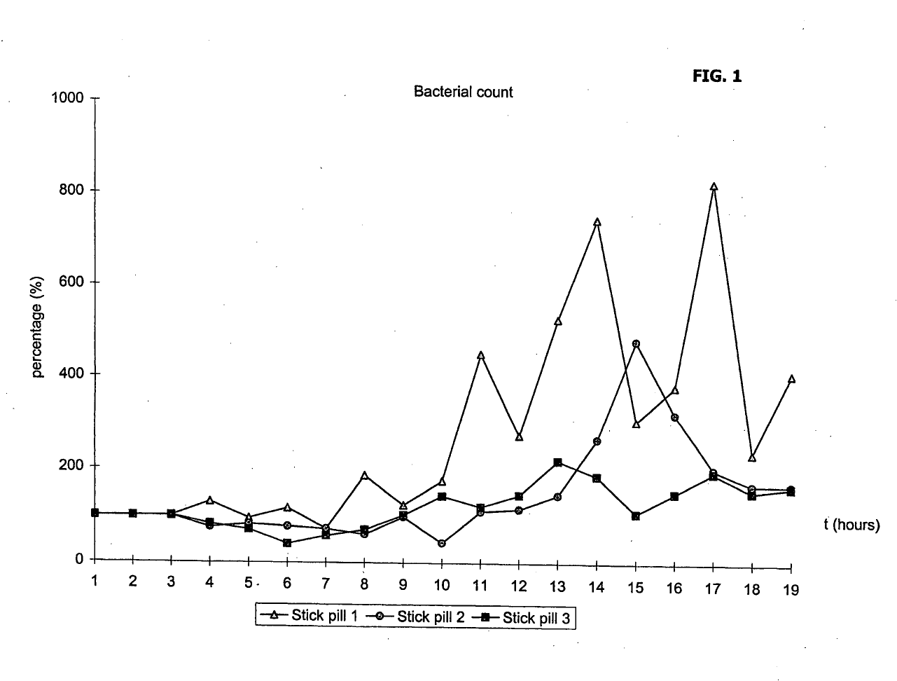

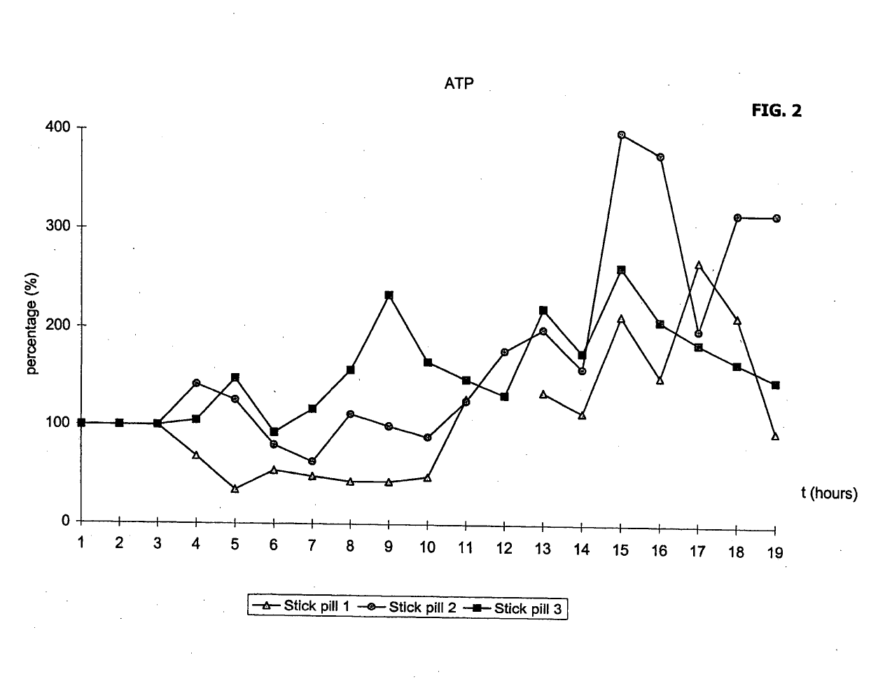

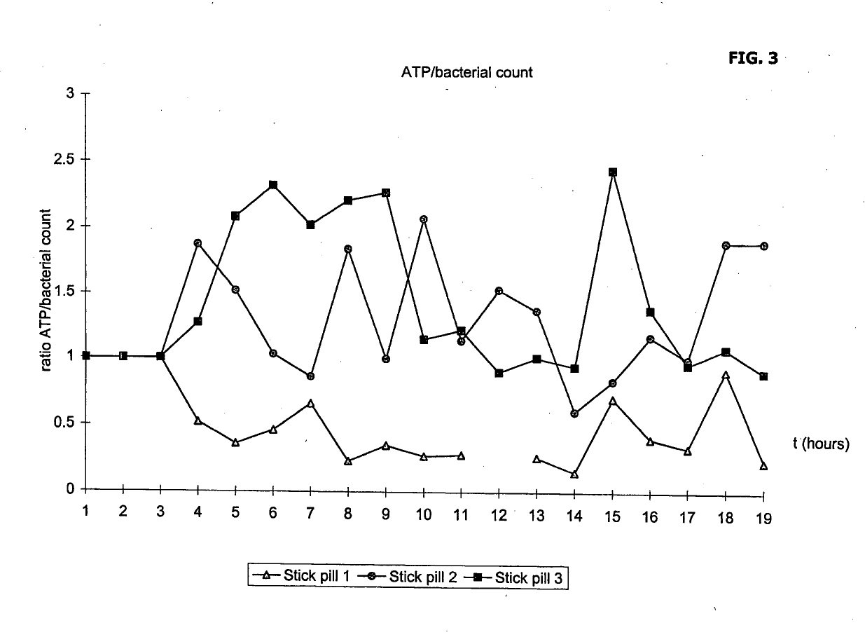

The saliva samples were taken off at different places of the mouth for each patient such as under the tongue, close to the crevicular canals). Each sample was divided in two parts: one to analyze the total microbial count on a Petri dish, the other one to analyze the ATP content of the microorganisms. The results are shown in FIGS. 1 and 2. As could be observed in FIG. 1, the microbial count is more important for the Stick 1 than for the Stick 2 and Stick 3. It could be reasoned that the presence of the growth factors can facilitate the growth of the bacteria in the saliva. When the ATP energy of these microorganisms contained in the samples was analyzed, it was difficult to observe important differences between the three groups (FIG. 2). On the other hand, when the ratio ATP/microbial count was analyzed, the results show an important difference characterized by much lower values for the Stick 1 compared to the Stick 2 and Stick 3 (FIG. 3).

Regarding the values of the microbial count (FIG. 1), it could be expected to find lower values for Stick 1 and Stick 2 containing the active ingredients. Observing the ratio ATP/microbial count (FIG. 3), it can be concluded that if the microbial count is higher for the Stick 1, it is due to the detachment of the bacterial biofilms linked to the mucosa cells which allows these isolated bacteria to be attacked and inhibited by the enzyme group. In the case of Stick 2, the action of the enzyme group is performed only on the isolated bacteria contained in the saliva but it is evident that regarding the microbial count and ATP energy values for this experiment, the enzyme group alone has no effect on the bacteria colonized in biofilms. This corresponds to the conclusions made by different researchers, which observe an action of the enzyme group in some experiments and not in other experiments because they never compared the difference between the isolated bacteria and the bacteria colonized in biofilms.

Eventually for Stick 3, as a control, it can be concluded that few bacteria can be eliminated by the saliva and certainly not the ones which are colonized under biofilm forms.

### Example VI

The same experiment as described in Example V was performed using a toothpaste containing the enzyme group and the growth factor group (Toothpaste 1) which was compared to a toothpaste containing only the enzyme group (Toothpaste 2) and a toothpaste containing no enzyme group and no growth factor group (Toothpaste 3).

Toothpaste with active ingredients Enzymes and Growth Factors: Toothpaste 1

| | Amounts per | |
|---|---|---|
| | 100 g of paste | |
| Active ingredients | | |
| Lactoperoxidase | 10,000 | ABTS units |
| Glucose Oxidase | 10,000 | ABTS units |
| Substrate (thiocyanate) | 50 | mg |
| Lactoferrin | 100 | mg |
| Lysozyme | 100 | mg |
| Immunoglobulins | 300 | mg |
| Derived Platelet growth factor | 100 | ng |
| Transforming growth factor | 120 | ng |
| Fibroblast growth factor | 50 | ng |
| Epidermal growth factor | 1 | ng |
| Angiogenin | 1 | ng |
| Non active ingredients: | | |
| Sorbitol, glycerin, silica, carboxymethylcellulose, | | |
| sodium benzoate, xylitol, flavor, titane dioxide | | |

Toothpaste with only the Enzymes as active ingredients: Toothpaste 2

| | Amounts per 100 g | |
|---|---|---|
| | of paste | |
| Active ingredients: | | |
| Lactoperoxidase | 10,000 | ABTS units |
| Glucose Oxidase | 10,000 | ABTS units |
| Substrate (thiocyanate) | 50 | mg |
| Lactoferrin | 100 | mg |
| Lysozyme | 100 | mg |
| Immunoglobulins | 300 | mg |
| Non active ingredients: | | |
| Sorbitol, glycerin, silica, carboxymethylcellulose, | | |
| sodium benzoate, xylitol, flavor, titane dioxide | | |

Toothpaste where the active ingredients are replaced by sorbitol: Toothpaste 3 (control)

Non Active Ingredients

Sorbitol, glycerin, silica, carboxymethylcellulose, sodium benzoate, xylitol, flavor, titane dioxide

The patients suffering from a dry mouth because they produced less than 0.2 ml per minute of saliva brushed their teeth for at least two minutes with the toothpastes 1 to 3. The patients rinsed their mouth with only 5 ml water and a sample is taken off. 24 patients were used in each group.

The saliva samples were taken off at different places of the mouth for each patient such as under the tongue, close to the crevicular canals. Each sample was divided in two parts: one to analyze the total microbial count on a Petri dish, the other one to analyze the ATP content of the microorganisms.

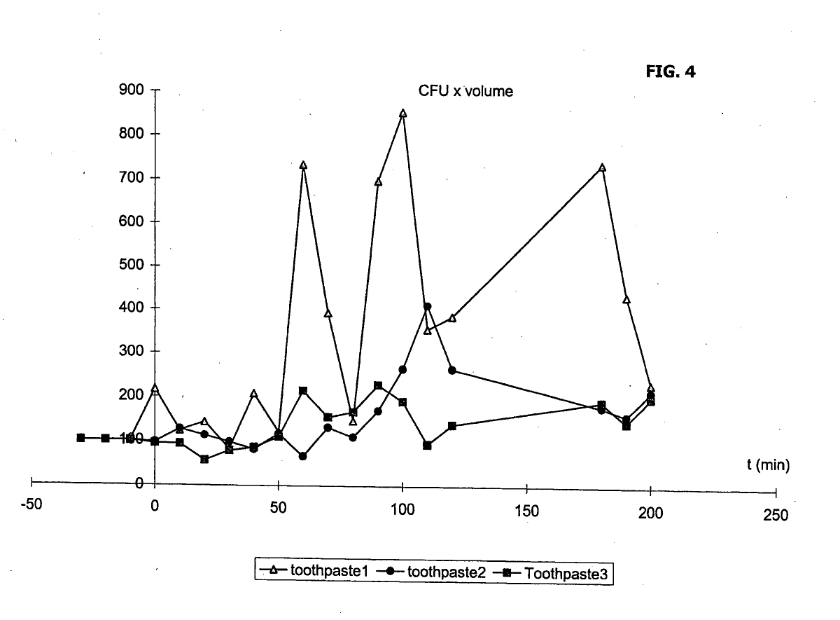

As can be observed, the microbial count is more important for the Toothpaste 1 than for the Toothpaste 2 and Toothpaste 3 (FIG. 4). It could be reasoned that the presence of the growth factors can facilitate the growth of the bacteria in the saliva.

When the ATP energy of these microorganisms contained in the samples was analyzed, it was difficult to observe important differences between the three groups.

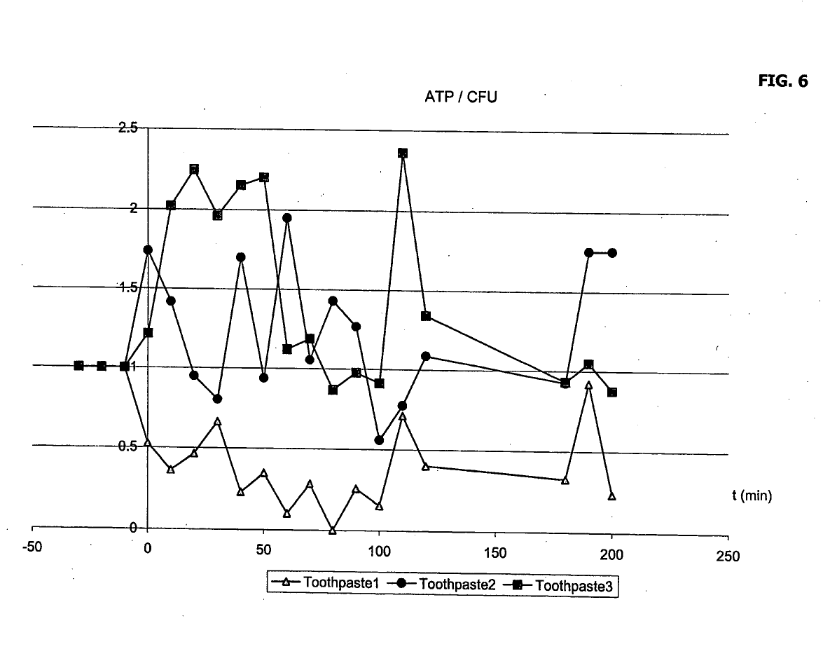

On the other hand, when the ratio ATP/microbial count is analyzed, the results show an important difference characterized by a much lower values for the Toothpaste 1 compared to the Toothpaste 2 and Toothpaste 3 (FIG. 6).

Regarding the values of the microbial count (FIG. 4), it could be expected to find lower values for the Toothpaste 1 and Toothpaste 2 containing the active ingredients. Observing the ratio ATP/microbial count (FIG. 6), it can be concluded that if the microbial count is higher for the Toothpaste 1, it is due to the detachment of the bacterial biofilms linked to the mucosa cells and allow to these isolated bacteria to be attacked and inhibited by the enzyme group. In the case of the Toothpaste 2, the action of the enzyme group is performed only on the isolated bacteria contained in the saliva but it is evident that regarding the microbial count and ATP energy values for this experiment, the enzyme group alone has no effect on the bacteria colonized in biofilms. This corresponds to the conclusions made by different researchers, which observe an action of the enzyme group in some experiments and not in other experiments because they never compared the difference between the isolated bacteria and the bacteria colonized in biofilms.

Eventually for the Toothpaste 3, as control, we can conclude that few bacteria can be eliminated by the saliva and certainly not the ones which are colonized under biofilm forms.

Example VII

| Sterilant solution | concentrated powder | |
|---|---|---|
| Lactoferrin peptides | 100 | mg |
| Epidermal factor | 10 | μg |
| Dialkyldimethylammonium chloride | 5 | g |

### Example VIII

| Sterilant solution | concentrated tablet | |
|---|---|---|
| Lactoferrin peptides | 250 | μg |
| Bovine whey colostrum | 10 | mg |
| Sodium bicarbonate | 1000 | mg |
| Polyvinylpyrrolidone | 50 | mg |
| Tartric acid | 1000 | mg |

The bovine whey colostrum results of the action of skimmed colostrum and calf rennet. This action precipitates some proteins from the colostrum. The supernatant is the whey.

### Example IX

| Sterilant solution | concentrated tablet |
|---|---|
| Lactoferrin | 1 mg |
| Bovine whey colostrum | 10 mg |
| Propylene glycol | 989 mg |

Lactoferrin and bovine whey colostrum is dissolved in propylene glycol to give 1 g of concentrated sterilant solution. 1 g of concentrated sterilant solution is dispensed from a measured dose bottle, measured dose pump pack, polymer or glass phial to be diluted with 90 ml of water to give a sterilant solution.

### Example X

### Toothpaste Tablets

| | | |
|---|---|---|
| Flavor | 0.06 | mg |
| Sodium fluoride | 0.15 | mg |
| Micro-silicium | 4.5 | mg |
| Lactoferrin peptides | 5 | mg |
| Bovine colostrum | 15 | mg |
| Amorphous silicium | 22.5 | mg |
| Mg stearate | 52.5 | mg |
| Xylitol | 600 | mg |
| Sorbitol DC 60/W | 739.515 | mg |

Considering that bovine colostrum contains the growth factors, the activity of this toothpaste is due to a synergistic effect between lactoferrin peptides and the bovine colostrum.

### Example XI

Illustrative formulations for pharmaceutically-acceptable carriers for the pharmaceutical composition of the present invention to be formulated as an aqueous cream for cosmetic use are set forth in the following tables, as follows. The following tables show the varying ingredients and the prophylactic and therapeutic effective amounts (quantities) which can be used in the preparation of an aqueous cream, according to the present invention.

Aqueous Cream for Cosmetic Use (a)

| | | |
|---|---|---|
| Lactoferrin peptides | 10 | mg |
| Bovine whey colostrum | 100 | mg |
| Emulsifying wax | 9 | g |
| Liquid paraffin | 6 | g |
| White soft paraffin | 10 | g |
| Purified water | to 100 | g |

Aqueous Cream for Cosmetic Use (b)

| | | | | |
|---|---|---|---|---|
| Lactoferrin peptides | 10 | mg | 10 | mg |
| Bovine whey colostrum | 100 | mg | 100 | mg |
| Emulsifying wax | 9 | g | 9 | g |
| White soft paraffin | 10 | g | 10 | g |
| OSCN- (500 μM in purified water) | to 100 | g | | |
| OI—+ OSCN- (450 μM + 50 μM in | | | to 100 | g |
| purified water respectively) | | | | |

### Example XII

Enteric-Coated Tablet

| | | |
|---|---|---|
| Hydroxypropyl methylcellulose phthalate | 6 | mg |
| Bovine whey colostrum | 100 | mg |
| Lactoferrin peptides | 1 | mg |
| Cetyl alcohol | 0.5 | mg |

The enteric coating solution is for example applied on the whey colostrum core by fluidized bed coating.

1. A composition comprising
(a) at least one ion selected from hypohalite, hypothiocyanite or a combination thereof,
(b) at least one compound selected from lactoferrin, lactoferrin peptide, lysozyme, immunoglobulins or a combination thereof, and
(c) at least one growth factor.

2. The composition according to claim 1, further comprising a compound selected from triclosan, chlorhexidine, cetylpyridinium, or a combination thereof.

3. The composition according to claim 1, wherein said hypohalite is selected from hypochlorite, hypoiodite, hypobromite or a combination thereof.

4. The composition according to claim 1, wherein said growth factor is selected from platelet derived growth factor, fibroblast growth factor, transforming growth factor, angiogenin, epidermal growth factor or a combination thereof.

5. The composition according to claim 1, wherein the composition comprises whey colostrum and/or colostrum as a source for said at least one growth factor.

6. The composition according to claim 1, wherein said at least one ion is in a concentration ranging from 0.1 μM to 5000 μM.

7. The composition according to claim 1, wherein said compound is lactoferrin, preferably in a concentration ranging from 0.1 μg to 5000 mg/l.

8. The composition according to claim 1, wherein said compound is lactoferrin peptide, preferably in a concentration ranging from 0.1 μg to 5000 mg/l.

9. The composition according to claim 1, wherein said compound is lysozyme, preferably in a concentration ranging from 0.1 μg to 5000 mg/l.

10. The composition according to claim 1, wherein said compound is an immunoglobulin, preferably in a concentration ranging from 0.1 μg to 100 g/l.

11. The composition according to claim 1, wherein said composition is fluid.

12. A pharmaceutical composition comprising a composition according to claim 1, and a pharmaceutically acceptable carrier.

13. A method for the prophylaxis and/or therapy of diseases caused by microorganisms comprising administering to an individual in need thereof a composition according to claim 1.

14. The method according to claim 13, wherein said microorganisms are bacteria, viruses, protozoas, yeast, fungi, parasites and the like

15. A method for preventing or treating diseases selected from gastrointestinal infections, calculus and occurrence of caries, gingivitis, mucositis, periodontal diseases, vaginal diseases, herpes, acne, ulcers, tourista, cystitis, molluscum contagiosum or chlamydia infections, comprising the step of administering to an individual in need thereof a composition according to claim 1.

16. A method for prophylaxis and therapy of infectious diseases caused by microorganisms present in biofilms adherent to cell surfaces comprising the step of administering to an individual in need thereof a composition according to claim 1.

17. The method for prophylaxis and therapy of infectious diseases caused by microorganisms present in biofilms adherent to cell surfaces comprising the step of administering to an individual in need thereof a composition comprising a combination of:
(a) at least one compound chosen from the group of peroxidase system, lactoferrin, lactoferrin peptides, lysozyme and immunoglobulins and
(b) at least one growth factor.

18. The method according to claim 17, wherein said growth factor is chosen from platelet derived growth factor, fibroblast growth factor, transforming growth factor, angiogenin and epidermal growth factor.

19. The method according to claim 17, wherein at least a peroxidase system is combined with a growth factor.

20. The method according to claim 17, wherein at least a lactoferrin is combined with a growth factor.

21. The method according to claim 17, wherein at least lysozyme is combined with a growth factor.

22. The method according to claim 17, wherein at least immunoglobulin is combined with a growth factor.

23. The method according to claim 17, wherein a lactoferrin peptide is combined with a growth factor.

24. The method according to claim 17, wherein said growth factor is platelet derived growth factor.

25. The method according to claim 17, wherein said growth factor is fibroblast growth factor.

26. The method according to claim 17, wherein said growth factor is transforming growth factor.

27. The method according to claim 17, wherein growth factor is epidermal growth factor.

28. The method according to claim 17, wherein said growth factor is angiogenin.

29. The method according to claim 17, wherein the composition comprises whey colostrum and/or colostrum as a source for said at least one growth factor.

30. The method according to claim 17, wherein said composition is in the form of a gel, a stick pill, a rinsing liquid, a toothpaste, a tablet, a topical medicament, an oral dentifrice, an injectable composition, an oral tablet, a lozenge or a soft gelatin capsule.

31. The method according to claim 17, wherein the composition is combined with a pharmaceutically acceptable carrier.

32. A method for prophylaxis or therapy of infectious diseases in humans or animals caused by microorganisms present in biofilms adherent to cell surfaces comprising:
(a) treating said human or animal with a growth factor, preferably chosen from the group of platelet derived growth factor, fibroblast growth factor, transforming growth factor, angiogenin and epidermal growth factor used alone or in combination; and,
(b) treating said human or animal with at least one compound chosen from the group of peroxidase, lactoferrin, lactoferrin peptides, lysozyme and immunoglobulin used alone or in combination.
